(19) United States
(12) Patent Application Publication
(10) Pub. No.: US 2009/0197958 A1
(43) Pub. Date: Aug. 6, 2009

(54) Sustained Release Particulate Oral Dosage Forms of (R) Baclofen and Methods of Treatment

(75) Inventors: Srikonsda SASTRY, Sunnyvale, CA (US); Kenneth C. Cundy, Redwood City, CA (US); Manshiu Leung, Daly City, CA (US)

Correspondence Address: DORSEY & WHITNEY, LLP;INTELLECTUAL PROPERTY DEPARTMENT, 370 SEVENTEENTH STREET, SUITE 4700, DENVER, CO, 80202-5647, US

(73) Assignee: XenoPort, Inc., Santa Clara, CA (US)

(21) Appl. No.: 12/024,830

(22) Filed: Feb. 1, 2008

(51) Int. Cl. A61K 31/195; A61P 25/32; A61P 25/34; A61P 25/36; A61P 25/02; A61P 1/04; A61P 1/08

(52) U.S. Cl. 514/563; 514/567

(57) ABSTRACT

Sustained release particulate oral dosage forms of (R)-baclofen prodrugs and methods of treating a disease comprising orally administering such dosage forms are disclosed.

### FIELD

The present disclosure relates to sustained release particulate oral dosage forms of (R)-baclofen prodrugs and methods of treating a disease comprising orally administering such dosage forms.

### BACKGROUND

(±)-4-Amino-3-(4-chlorophenyl)butanoic acid (baclofen), (1), is an analog of gamma-aminobutyric acid (i.e., GABA) that selectively activates GABA_{B} receptors, resulting in neuronal hyperpolarization. GABA_{B} receptors are located in laminae I-IV of the spinal cord, where primary sensory fibers end. These G-protein coupled receptors activate conductance by K⁺-selective ion channels and can reduce currents mediated by Ca²⁺ channels in certain neurons. Baclofen has a pre-synaptic inhibitory effect on the release of excitatory neurotransmitters and also acts postsynaptically to decrease motor neuron firing (see Bowery, Trends Pharmacol. Sci. 1989, 10, 401-7; and Misgeld et al., Prog. Neurobiol. 1995, 46, 423-62).

A principal pharmacological effect of baclofen in mammals is reduction of muscle tone and the drug is frequently used in the treatment of spasticity (Price et al., nature 1984, 307, 71-4). Spasticity is associated with damage to the corticospinal tract and is a common complication of neurological disease. Diseases and conditions in which spasticity may be a prominent symptom include cerebral palsy, multiple sclerosis, stroke, head and spinal cord injuries, traumatic brain injury, anoxia, and neurodegenerative diseases. Patients with spasticity complain of stiffness, involuntary spasm, and pain. These painful spasms may be spontaneous or triggered by a minor sensory stimulus, such as touching the patient.

Baclofen is also useful in controlling gastro-esophageal reflux disease (van Herwaarden et al., Aliment. Pharmacol. Ther. 2002, 16, 1655-62; Ciccaglione et al., Gut 2003, 52, 464-70; Andrews et al., U.S. Pat. No. 6,117,908; and Fara et al., International Publication No. WO 02/096404); in promoting alcohol abstinence in alcoholics (Gessa et al., International Publication No. WO 01/26638); in promoting smoking cessation (Gessa et al., International Publication No. WO 01/08675); in reducing addiction liability of narcotic agents (Robson et al., U.S. Pat. No. 4,126,684); in the treatment of emesis (Bountra et al., U.S. Pat. No. 5,719,185); as an anti-tussive for the treatment of cough (Kreutner et al., U.S. Pat. No. 5,006,560); in treating neuropathic pain such as trigeminal neuralgia (Bowsher, Br. Med. Bull. 1991, 47(3), 655-66; Fromm et al., Neurology 1981, 31, 683-7; and Ringel and Roy, Ann Neurol 1987, 21(5), 514-5); and in treating musculoskeletal pain such as low back pain (Dapas et al., Spine 1985, 10(4), 345-9; and Raphael et al., BMC Musculoskeletal Disorders 2002, 3(17), Epub 2002 Jun. 20); tension-type headaches (Freitag, CNS Drugs 2003, 17(6), 373-81); and radiculopathy (Zuniga et al., Anesthesiology 2000, 92(3), 876-880).

Baclofen may be administered orally or by intrathecal delivery through a surgically implanted programmable pump. The drug is rapidly absorbed from the gastrointestinal tract and has an elimination half-life of approximately 3-4 hours. Baclofen is partially metabolized in the liver but is largely excreted by the kidneys unchanged. The short half-life of baclofen necessitates frequent administration with typical oral dosing regimens ranging from about 10 to about 80 mg of three or four divided doses daily. Plasma baclofen concentrations of about 80 to about 400 ng/mL result from these therapeutically effective doses in patients (Katz, Am. J. Phys. Med. Rehabil. 1988, 67(3), 108-16; and Krach, J. Child Neurol. 2001, 16(1), 31-6). When baclofen is given orally, sedation is an adverse effect, particularly at elevated doses. Impairment of cognitive function, confusion, memory loss, dizziness, weakness, ataxia, and orthostatic hypotension are other commonly encountered adverse effects of baclofen therapy.

Intrathecal administration is often recommended for patients who find the adverse effects of oral baclofen intolerable. The intrathecal use of baclofen permits effective treatment of spasticity with doses less than 1/100^{th} of those required orally, since administration directly into the spinal subarachnoid space permits immediate access to the GABA_{B} receptor sites in the dorsal horn of the spinal cord. Surgical implantation of a pump is, however, inconvenient and a variety of mechanical and medical complications can arise (e.g., catheter displacement, kinking or blockage, pump failure, sepsis, and deep vein thrombosis). Acute discontinuation of baclofen therapy (e.g., in cases of mechanical failure) may cause serious withdrawal symptoms such as hallucinations, confusion, agitation, and seizures (Sampathkumar et al., Anesth. Analg. 1998, 87, 562-63).

While the clinically prescribed baclofen product (Lioresal™) is available only as a racemate, the GABA_{B} receptor agonist activity resides entirely in one enantiomer, R-(−)-baclofen (2) (also termed L-baclofen).

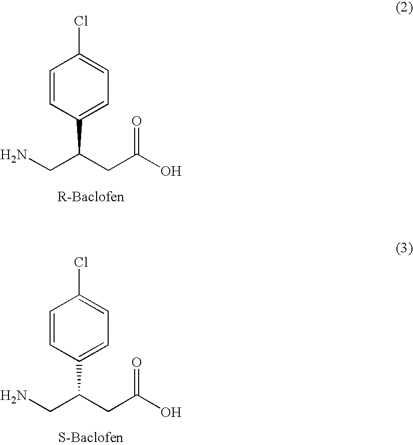

The other isomer, (S)-baclofen, (3), actually antagonizes the action of (R)-baclofen at GABA_{B} receptors and exhibits antinociceptive activity in the rat spinal cord (Sawynok et al., Pharmacology 1985, 31, 248-59). Orally administered (R)-baclofen is reported to be about 5-fold more potent than orally administered racemic baclofen, with an (R)-baclofen regimen of 2 mg t.i.d being equivalent to racemic baclofen at 10 mg t.i.d. (Fromm et al., Neurology 1987, 37, 1725-8). Moreover, the adverse effect profile following administration of R-baclofen has been shown to be significantly reduced relative to an equally efficacious dose of racemic baclofen.

Baclofen, a zwitterionic amino acid, lacks the requisite physicochemical characteristics for effective passive permeability across cellular membranes. Passage of the drug across the gastrointestinal tract and the blood-brain barrier (BBB) are mediated primarily by active transport processes rather than by passive diffusion. Accordingly, baclofen is a substrate for active transport mechanisms shared by neutral α-amino acids such as leucine, and β-amino acids such as β-alanine and taurine (van Bree et al., Pharm. Res. 1988, 5(6), 369-71; Cercos-Fortea et al., Biopharm. Drug. Disp. 1995, 16, 563-77; Deguchi et al., Pharm. Res. 1995, 12(12), 1838-44; and Moll-Navarro et al., J. Pharm. Sci. 1996, 85(11), 1248-54). Transport across the BBB is stereoselective, with preferential uptake of the active R-enantiomer (2) being reported (van Bree et al., Pharm. Res. 1991, 8(2), 259-62). In addition, organic anion transporters localized in capillary endothelial cells of the blood-brain barrier have been implicated in efflux of baclofen from the brain (Deguchi et al, Pharm. Res. 1995, 12(12), 1838-44; and Ohtsuki et al., J. Neurochem. 2002, 83, 57-66). 3-(p-Chlorophenyl)pyrrolidine has been described as a CNS-penetrable prodrug of baclofen (Wall et al., J. Med. Chem. 1989, 32, 1340-8). Other prodrugs of (R)-baclofen are described in Bryans et al, International Publication No. WO 01/90052; Bryans et al. EP1178034; Cundy et al., U.S. Pat. No. 6,992,076; Gallop et al, U.S. Pat. Nos. 6,818,787, 6,927,036, and 6,972,341; and Raillard et al., U.S. Pat. No. 7,232,924.

Sustained released oral dosage formulations are a conventional solution to the problem of rapid systemic drug clearance, as is well known in the art (see, e.g., “The Science and Practice of Pharmacy,” 21^{st} Ed., Lippincott Williams & Wilkins, 2005, Chapters 46-47). Osmotic delivery systems are also recognized methods for sustained drug delivery (see, e.g., Verma et al., Drug Dev. Ind Pharm. 2000, 26, 695-708). Successful application of these technologies depends on the drug of interest having an effective level of absorption from the large intestine (also referred to herein as the colon) where the dosage form spends a majority of its time during its passage down the gastrointestinal tract. Baclofen is poorly absorbed following administration into the colon in animal models (Merino et al., Biopharm. Drug. Disp. 1989, 10(3), 279-97) presumably because the transporter proteins mediating baclofen absorption in the upper region of the small intestine are not expressed in the large intestine. Development of an oral controlled release formulation for baclofen should considerably improve the convenience, efficacy, and adverse effect profile of baclofen therapy. However, the rapid passage of conventional dosage forms through the proximal absorptive region of the small intestine has thus far prevented the successful application of sustained release technologies to this drug.

Recently, Gallop et al. have developed new prodrugs of (R)-baclofen and baclofen analogs that are well absorbed in the large intestine/colon and hence suitable for oral sustained release formulations, thus improving the convenience, efficacy and side effect profile of baclofen therapy (Gallop et al., U.S. Pat. Nos. 7,109,239 and 7,227,028, U.S. Application Publication No. 2007/0054945, and U.S. Application Publication No. 2007/0244331; Leung et al., U.S. Provisional Application No. 60/884,598 filed Jan. 11, 2007; and Cundy, U.S. Provisional Application No. 60/944,475 filed Jun. 15, 2007; each of which is incorporated by reference herein in its entirety. For example, (3R)-4-{[(1S)-2-methyl-1-(2-methylpropanoyloxy)propoxy]carbonylamino}-3-(4-chlorophenyl)butanoic acid, (4),

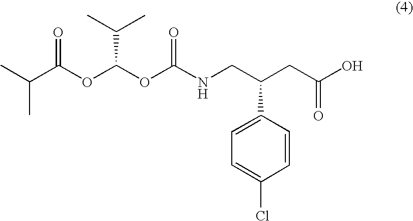

a prodrug of the GABA analog, (R)-baclofen (2) ((±)-4-amino-3-(4-chlorophenyl)butanoic acid) (1), exhibits high bioavailability as (R)-baclofen when dosed either orally or directly into the colon of a mammal (Gallop et al., U.S. Pat. Nos. 7,109,239).

The high oral bioavailability indicates the efficacious use of compound (4) in oral dosage forms, including sustained-release dosage forms, useful for treating a disease in which baclofen is known to be effective such as spasticity and gastro-esophageal reflux disease (van Herwaarden et al., Aliment. Pharmacol. Ther. 2002, 16, 1655-62; Ciccaglione et al., Gut 2003, 52, 464-70; Andrews et al., U.S. Pat. No. 6,117,908; and Fara et al., International Publication No. WO 02/096404); in promoting alcohol abstinence in alcoholics (Gessa et al., International Publication No. WO 01/26638); in promoting smoking cessation (Gessa et al., International Publication No. WO 01/08675); in reducing addiction liability of narcotic agents (Robson et al., U.S. Pat. No. 4,126,684); in the treatment of emesis (Bountra et al, U.S. Pat. No. 5,719,185); as an anti-tussive for the treatment of cough (Kreutner et al., U.S. Pat. No. 5,006,560); in the treatment of neuropathic pain such as trigeminal neuralgia (Bowsher, Br. Med. Bull. 1991, 47(3), 655-66; Fromm et al., Neurology 1981, 31, 683-7; and Ringel and Roy, Ann Neurol 1987, 21(5), 514-5), in the treatment of musculoskeletal pain such as low back pain (Dapas et al., Spine 1985, 10(4), 345-9; and Raphael et al., BMC Musculoskeletal Disorders 2002, 3(17), Epub 2002 Jun. 20; as well as for treating movement disorders such as dystonia and hiccups; peripheral nerve disorders such as muscle stimulation disorders; spinal cord disorders such as spastic paraparesis; multiple sclerosis; and cerebral palsy.

Controlled release tablet dosage forms comprising (3R)-4-{[(1S)-2-methyl-1-(2-methylpropanoyloxy)propoxy]carbonylamino}-3-(4-chlorophenyl)butanoic acid are disclosed by Leung et al., U.S. Provisional Application No. 60/884,598, filed Jan. 11, 2007, which is incorporated by reference herein in its entirety.

### SUMMARY

(R)-baclofen prodrugs having an enhanced oral bioavailability profile and that are well absorbed in the large intestine/colon can be used in oral sustained release dosage forms to improve the convenience, efficacy, and adverse effect profile of (R)-baclofen therapy.

In a first aspect, oral pharmaceutical dosage forms of an (R)-baclofen prodrug are provided, comprising at least two particle populations, wherein at least one of the two particle populations is chosen from (a) and (b): (a) a population of (R)-baclofen prodrug-containing particles, the particles when placed in an aqueous solution releasing the (R)-baclofen prodrug into the solution with a release profile that is independent of the solution pH; and (b) a population of (R)-baclofen prodrug-containing particles, the particles when placed in an aqueous solution releasing the (R)-baclofen prodrug into the solution with a release profile that is dependent on the solution pH; wherein the oral dosage form provides a therapeutically effective concentration of (R)-baclofen in the blood and/or plasma of a patient for a continuous period of time after the oral dosage form is orally administered to the patient.

In a second aspect, oral pharmaceutical dosage forms of (3R)-4-{[(1S)-2-methyl-1-(2-methylpropanoyloxy)propoxy]carbonylamino}-3-(4-chlorophenyl)butanoic acid (4), a pharmaceutically acceptable salt thereof, or a pharmaceutically acceptable solvate of any of the foregoing are provided, which when administered orally to a patient provides a therapeutically effective concentration of (R)-baclofen in the blood and/or plasma of the patient for a continuous period of time after the dosage form is orally administered to the patient, wherein the concentration of (R)-baclofen in the blood plasma of the patient does not exceed a minimum adverse concentration at any time after the dosage form is orally administered to the patient.

In a third aspect, oral dosage forms are provided comprising a plurality of particles, and at least one pH independent release controlling polymer, which following oral administration to a human patient provides a blood (R)-3-amino-3-(4-chlorophenyl)butanoic acid concentration characterized by a Cₘₐₓ/C₁₂ ratio ranging from about 1 to about 6, a Cₘₐₓ/dose ratio ranging from about 1.25 (10⁶·mL)⁻¹ to about 3.25 (10⁶·mL)⁻¹, and an AUC_{inf}/dose ratio ranging from about 13 (hr/10⁶·mL) to about 33 (hr/10⁶·mL).

In a fourth aspect, methods of treating a disease such as spasticity, gastro-esophageal reflux disease, emesis, cough, narcotic addiction or abuse, alcohol addiction or abuse, nicotine addiction or abuse, neuropathic pain, or musculoskeletal pain in a patient are provided comprising orally administering to a patient in need of such treatment a pharmaceutical dosage form comprising at least one population of controlled-release particles comprising an (R)-baclofen prodrug or a pharmaceutically acceptable salt or solvate thereof.

These and other features provided by the present disclosure are set forth herein.

### BRIEF DESCRIPTION OF THE DRAWINGS

The skilled artisan will understand that the drawings, described herein, are for illustration purposes only. The drawings are not intended to limit the scope of the present disclosure.

FIG. 1 shows an in vitro dissolution profile of a dosage form comprising immediate release particles (population (c) particles).

FIG. 2 shows an in vitro dissolution profile of a dosage form comprising pH-dependent release particles (population (b) particles).

FIG. 3 shows an in vitro dissolution profile of a dosage form comprising pH-independent release particles (population (a) particles).

FIG. 4 shows the concentration of (R)-baclofen in the plasma of fed healthy human patients following oral administration of capsule dosage forms containing particles comprising compound (4).

FIG. 5 shows the concentration of (R)-baclofen in the plasma of fasted healthy human patients following oral administration of capsule dosage forms containing particles comprising compound (4).

FIG. 6 shows the mean (SD) concentration of (R)-baclofen in blood of healthy human patients following oral administration of controlled release capsules at doses of 10, 20, 30, 40, 60, and 80 mg compound (4).

FIG. 7 shows the relationship between the dose of compound (4) and the Cₘₐₓ of (R)-baclofen in blood of healthy human patients following oral administration of controlled release capsules.

FIG. 8 shows the relationship between the dose of compound (4) and the AUC_{inf} of (R)-baclofen in blood of healthy human patients following oral administration of controlled release capsules.

FIG. 9 shows the mean concentrations of compound (4) and (R)-baclofen in blood of healthy human patients following oral administration of controlled release capsules comprising 10 mg compound (4) at a dose of 80 mg compound (4).

### DETAILED DESCRIPTION

### Definitions

A dash (“-”) that is not between two letters or symbols is used to indicate a point of attachment for a substituent. For example, —CONH₂ is attached through the carbon atom.

“Adverse drug effects” refers to drug effects that are unwanted, unpleasant, noxious, and/or potentially harmful. Adverse drug effects can be mild such as digestive disturbance, headaches, fatigue, vague muscle aches, malaise, and changes in sleep patterns. Moderate adverse drug effects represent reactions that a person experiencing them considers annoying, distressing, or intolerable such as skin rashes, visual disturbances, muscle tremor, difficulty with urination, perceptible changes in mood or mental function, and/or certain changes in blood components. Examples of severe adverse drug effects include reactions that may be life threatening, that result in persistent or significant disability or hospitalization, and/or that cause a birth defect. Examples of adverse effects known to be associated with baclofen therapy include sedation, impairment of cognitive function, confusion, memory loss, dizziness, weakness, ataxia, blurred or double vision, nausea, shortness of breath, convulsions, and orthostatic hypotension.

“Alkyl” by itself or as part of another substituent refers to a saturated or unsaturated, branched, or straight-chain monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl(allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, an alkyl group comprises from 1 to 20 carbon atoms, in certain embodiments, from 1 to 10 carbon atoms, and in certain embodiments, from 1 to 8 or 1 to 6 carbon atoms.

“Acyl” by itself or as part of another substituent refers to a radical —C(O)R⁷⁰, where R⁷⁰ is hydrogen, alkyl, heteroalkyl, cycloalkyl, heterocycloalkyl, cycloalkylalkyl, heterocycloalkylalkyl, aryl, heteroaryl, arylalkyl, or heteroarylalkyl, which can be substituted, as defined herein. Examples of acyl groups include, but are not limited to, formyl, acetyl, cyclohexylcarbonyl, cyclohexylmethylcarbonyl, benzoyl, benzylcarbonyl, and the like.

“Alkoxy” by itself or as part of another substituent refers to a radical —OR⁷¹ where R⁷¹ is alkyl, cycloalkyl, cycloalkylalkyl, aryl, or arylalkyl, which can be substituted, as defined herein. In some embodiments, alkoxy groups have from 1 to 8 carbon atoms. Examples of alkoxy groups include, but are not limited to, methoxy, ethoxy, propoxy, butoxy, cyclohexyloxy, and the like.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 5 to 20 carbon atoms, and in certain embodiments, from 5 to 12 carbon atoms. Aryl, however, does not encompass or overlap in any way with heteroaryl, separately defined herein. Hence, a multiple ring system in which one or more carbocyclic aromatic rings is fused to a heterocycloalkyl aromatic ring, is heteroaryl, not aryl, as defined herein.

“Arylalkyl” by itself or as part of another substituent refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound in a biological fluid in a patient as a function of time following administration of the compound to the patient. Examples of biological fluids include plasma and blood. The AUC can be determined by measuring the concentration of a compound in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. As relevant to the disclosure herein, an AUC for a (R)-baclofen prodrug can be determined by measuring the concentration of (R)-baclofen in the plasma or blood of a patient following oral administration of a dosage form comprising a prodrug of (R)-baclofen.

“Bioavailability” refers to the rate and amount of a drug that reaches the systemic circulation of a patient following administration of the drug or prodrug thereof to the patient and can be determined by evaluating, for example, the plasma or blood concentration-versus-time profile for a drug. Parameters useful in characterizing a plasma or blood concentration-versus-time curve include the area under the curve (AUC), the time to peak concentration (Tₘₐₓ), and the maximum drug concentration (Cₘₐₓ), where Cₘₐₓ is the maximum concentration of a drug in the plasma or blood of a patient following administration of a dose of the drug or form of drug to the patient, and Tₘₐₓ is the time to the maximum concentration (Cₘₐₓ) of a drug in the plasma or blood of a patient following administration of a dose of the drug or form of drug to the patient.

“Bioequivalence” refers to equivalence of the rate and extent of absorption of a drug after administration of equal doses of the drug or prodrug to a patient. As used herein, two pharmacokinetic profiles are bioequivalent if the 90% confidence interval for the ratio of the mean response of the two profiles is within the limits of 0.8 and 1.25. The mean response includes at least one of the characteristic parameters of a profile such as CₘₐₓTₘₐₓ, and AUC. Bioequivalence as used herein is consistent with the term as defined by the U.S. Food and Drug Administration and discussed in “Guidance for Industry—Bioavailability and Bioequivalence Studies for Orally Administered Drug Products” (2003).

“Cₘₐₓ” is the maximum drug concentration observed in the blood or plasma following administration of a dose of drug.

“Tₘₐₓ” is the time to the maximum drug concentration (Cₘₐₓ) following administration of a dose of drug.

“T_{1/2}” is the time interval between Tₘₐₓ and the time at which the drug concentration in the blood or plasma has decreased to one-half the maximum drug concentration.

Compounds encompassed by structural Formula (I) disclosed herein include any specific compounds within these formulae. Compounds may be identified either by their chemical structure and/or chemical name. When the chemical structure and chemical name conflict, the chemical structure is determinative of the identity of the compound. The compounds described herein may contain one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan.

Compounds of Formula (I) include, but are not limited to, optical isomers of compounds of Formula (I), racemates thereof, and other mixtures thereof. In such embodiments, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula (I) include Z- and E-forms (e.g., cis- and trans-forms) of compounds with double bonds.

In embodiments in which compounds of Formula (I) exist in various tautomeric forms, compounds provided by the present disclosure include all tautomeric forms of the compound. The compounds of Formula (I) may also exist in several tautomeric forms including the enol form, the keto form, and mixtures thereof. Accordingly, the chemical structures depicted herein encompass all possible tautomeric forms of the illustrated compounds. The compounds of Formula (I) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in single or multiple crystalline or amorphous forms. In general, all physical forms are equivalent for the uses contemplated herein and are intended to be within the scope of the present disclosure.

Further, when partial structures of the compounds are illustrated, an asterisk (*) indicates the point of attachment of the partial structure to the rest of the molecule.

“Colonically absorbable prodrug of (R)-baclofen” means a prodrug of (R)-baclofen, as defined herein, which provides an AUC of (R)-baclofen following colonic administration of the prodrug that is at least two times greater than the AUC of (R)-baclofen following colonic administration of an equivalent amount of (R)-baclofen itself.

“Controlled-release” refers to release of a drug from a dosage form in which the drug release is controlled or modified over a period of time. Controlled can mean, for example, sustained, delayed or pulsed-release at a particular time. Controlled can also mean that release of the drug from the dosage form is extended for longer than it would be in an immediate-release dosage form, i.e., at least over several hours.

“Controlled release capsules” refer to capsule dosage forms containing a plurality of particles comprising a (R)-baclofen prodrug and at least one controlled release polymer. In certain embodiments of controlled release capsules, the (R)-baclofen prodrug is (3R)-4-{[(1S)-2-methyl-1-(2-methylpropanoyloxy)propoxy]carbonylamino}-3-(4-chlorophenyl)butanoic acid, (4), a pharmaceutically acceptable salt thereof, or a pharmaceutically acceptable salt of any of the foregoing. In certain embodiments of controlled release capsules, the plurality of particles is a population of pH-independent release particles.

“Cycloalkyl” by itself or as part of another substituent refers to a partially saturated or unsaturated cyclic alkyl radical. Where a specific level of saturation is intended, the nomenclature “cycloalkanyl” or “cycloalkenyl” is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₃₋₁₂ cycloalkyl or C₅₋₁₂ cycloalkyl.

“Cycloalkylalkyl” by itself or as part of another substituent refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₇₋₃₀ cycloalkylalkyl, e.g., the alkanyl, alkenyl, or alkynyl moiety of the cycloalkylalkyl group is C₁₋₁₀ and the cycloalkyl moiety is C₆₋₂₀, and in certain embodiments, a cycloalkylalkyl group is C₇₋₂₀ cycloalkylalkyl, e.g., the alkanyl, alkenyl, or alkynyl moiety of the cycloalkylalkyl group is C₁₋₈ and the cycloalkyl moiety is C₄₋₂₀ or C₆₋₁₂.

“Disease” refers to a disease, disorder, condition, or symptom.

“Dosage form” refers to a form of a pharmaceutical formulation that contains an amount of active agent or precursor thereof, e.g., an (R)-baclofen prodrug, compound (4), and/or (R)-baclofen, sufficient to achieve a therapeutic effect upon administration to a patient. Examples of dosage forms include capsules, tablets, and liquid suspensions. An oral dosage form is intended to be administered to a patient via the mouth and swallowed. When the formulation is a tablet or capsule, the dosage form is one such tablet or capsule. A dose refers to one or more dosage forms administered at about the same time and that are intended to produce a therapeutic effect.

“Fasted patient” refers to a patient whose stomach is substantially free of food at the time a dose is administered to the patient and for at least 4 hours following administration. The time at which a patient's stomach becomes substantially free of food following a meal can depend on a number of factors including, for example, the size of the meal such as the number of calories, the content of the meal such as the fat content, the health of the patient, and the condition of the patient's gastrointestinal tract. The stomach of a healthy human subject is typically substantially free of food after about 4 to about 8 following ingestion of food. In certain embodiments, a fasted patient does not eat any food, but can ingest any amount of water or clear liquid, from about 10 hours prior to dosing until about 4 hours after dosing, drinks about 250 mL of water about 2 hours and about 1 hour prior to dosing and about 250 mL of water about 2 hours after dosing, eats a lunch about 4 hours after dosing, and eats a dinner about 10 hours after dosing.

“Fed patient” refers to a patient whose stomach contains food. In certain embodiments, a fed patient begins eating a test meal about 30 minutes prior to dosing and completes eating the test meal about 5 minutes prior to dosing, eats a lunch 4 hours after dosing, and eats a dinner about 10 hours after dosing. A test meal may comprise a high fat (about 50% of the total number of calories in the test meal) and high calorie (about 1,000 total calories) breakfast such as, for example, two eggs fried in butter, two strips of bacon, two slices of wheat toast with butter, four ounces of hash brown potatoes, and eight ounces of whole milk. A test meal may contain about 150 protein calories, 250 carbohydrate calories, and about 500 to 600 fat calories.

“Halogen” refers to a fluoro, chloro, bromo, or iodo group.

“Heteroalkyl” by itself or as part of another substituent refer to an alkyl group in which one or more of the carbon atoms (and any associated hydrogen atoms) are independently replaced with the same or different heteroatomic groups. In some embodiments, heteroalkyl groups have from 1 to 8 carbon atoms. Examples of heteroatomic groups include, but are not limited to, —O—, —S—, —O—O—, —S—S—, —O—S—, R⁷⁷R⁷⁸—, ═N—N═, —N═N—, —N═N—NR⁷⁹R⁸⁰, —PR⁸¹—, —P(O)₂—, —POR⁸²—, —O—P(O)₂—, —SO—, —SO₂—, —SnR⁸³R⁸⁴— and the like where R⁷⁷, R⁷⁸, R⁷⁹, R⁸⁰, R⁸¹, R⁸², R⁸³ and R⁸⁴ are independently hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, or substituted heteroarylalkyl. Where a specific level of saturation is intended, the nomenclature “heteroalkanyl,” “heteroalkenyl,” or “heteroalkynyl” is used.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which can be aromatic or non-aromatic in which at least one ring atom is a heteroatom. Heteroaryl encompasses 5- to 12-membered aromatic, such as 5- to 7-membered, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is from 5- to 20-membered heteroaryl, and in certain embodiments from 5- to 12-membered heteroaryl or from 5- to 10-membered heteroaryl. In certain embodiments heteroaryl groups are those derived from thiophene, pyrrole, benzothiophene, benzofuran, indole, pyridine, quinoline, imidazole, oxazole, and pyrazine.

“Heteroarylalkyl” by itself or as part of another substituent refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, or heteroarylalkynyl is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“Heterocycloalkyl” by itself or as part of another substituent refers to a partially saturated or unsaturated cyclic alkyl radical in which one or more carbon atoms (and any associated hydrogen atoms) are independently replaced with the same or different heteroatom. Examples of heteroatoms to replace the carbon atom(s) include, but are not limited to, N, P, O, S, Si, etc. Where a specific level of saturation is intended, the nomenclature “heterocycloalkanyl” or “heterocycloalkenyl” is used. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like.

“Prodrug of (R)-baclofen” or “prodrug of (R)-baclofen provided by the present disclosure” refers to a compound in which a promoiety that is cleavable in vivo, and is covalently bound to (R)-baclofen. In certain embodiments, a prodrug may be actively transported by transporters expressed in the enterocytes lining the gastrointestinal tract and/or be passively absorbed from the gastrointestinal tract. Prodrugs of (R)-baclofen can be stable in the gastrointestinal tract and following absorption are cleaved in the systemic circulation to release (R)-baclofen. In certain embodiments, a prodrug of (R)-baclofen provides a greater oral bioavailability of (R)-baclofen compared to the oral bioavailability of (R)-baclofen when administered as a uniform liquid immediate release formulation. In certain embodiments, a prodrug of (R)-baclofen provides a high oral bioavailability of (R)-baclofen, for example, exhibiting a (R)-baclofen oral bioavailability that is at least 10 times greater than the oral bioavailability of (R)-baclofen when orally administered in an equivalent dosage form. In certain embodiments, a prodrug of (R)-baclofen is a compound having a structure of Formula (I):

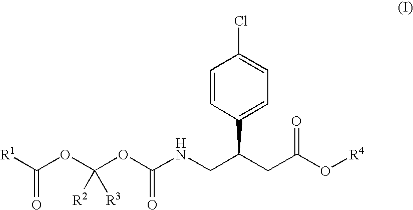

a pharmaceutically acceptable salts thereof, or a pharmaceutically acceptable solvate of any of the foregoing, wherein:

R¹ is chosen from acyl, substituted acyl, alkyl, substituted alkyl, aryl, substituted aryl, arylalkyl, substituted arylalkyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, and substituted heteroarylalkyl;

R² and R³ are independently chosen from hydrogen, alkyl, substituted alkyl, alkoxycarbonyl, substituted alkoxycarbonyl, aryl, substituted aryl, arylalkyl, substituted arylalkyl, cycloalkyl, substituted cycloalkyl, heteroaryl, substituted heteroaryl, heteroarylalkyl, and substituted heteroarylalkyl or; R² and R³ together with the carbon atom to which they are bonded form a ring chosen from a cycloalkyl, substituted cycloalkyl, heterocycloalkyl, and substituted heterocycloalkyl ring; and

R⁴ is chosen from hydrogen, alkyl, substituted alkyl, aryl, substituted aryl, arylalkyl, substituted arylalkyl, aryldialkylsilyl, cycloalkyl, substituted cycloalkyl, heterocycloalkyl, substituted heterocycloalkyl, heteroalkyl, substituted heteroalkyl, heteroaryl, substituted heteroaryl, heteroarylalkyl, substituted heteroarylalkyl, and trialkylsilyl.

In certain embodiments of a compound of Formula (I), the substituent group is independently chosen from halogen, hydroxyl, —COOH, —CN, and C₁₋₄ alkyl. In certain embodiments, each substituent group is independently chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

In certain embodiments of a compound of Formula (I), R¹ is chosen from methyl, ethyl, n-propyl, isopropyl, n-butyl, isobutyl, sec-butyl, tert-butyl, n-pentyl, isopentyl, sec-pentyl, neopentyl, 1,1-diethoxyethyl, phenyl, cyclohexyl, 2-pyridyl, 3-pyridyl, and 4-pyridyl; R² is chosen from hydrogen, methyl, ethyl, n-propyl, isopropyl, n-butyl, isobutyl, sec-butyl, phenyl, and cyclohexyl; R³ is hydrogen; and R⁴ is hydrogen.

In certain embodiments of a compound of Formula (I), R¹ is chosen from methyl, ethyl, n-propyl, isopropyl, n-butyl, isobutyl, sec-butyl, tert-butyl, phenyl, cyclohexyl, and 3-pyridyl; R² is hydrogen; R³ is hydrogen; and R⁴ is hydrogen.

In certain embodiments of a compound of Formula (I), R¹ is chosen from methyl, ethyl, n-propyl, isopropyl, n-butyl, isobutyl, sec-butyl, tert-butyl, phenyl, and cyclohexyl; R² is chosen from methyl, n-propyl, and isopropyl; R³ is hydrogen; and R⁴ is hydrogen.

In certain embodiments of a compound of Formula (I), the compound is substantially one diastereomer.

In certain embodiments of a compound of Formula (I), R¹ is chosen from methyl, ethyl, n-propyl, isopropyl, n-butyl, isobutyl, sec-butyl, tert-butyl, phenyl, and cyclohexyl; R² is isopropyl; R³ is hydrogen; and R⁴ is hydrogen.

In certain embodiments of a compound of Formula (I), R¹ is isopropyl; R² is isopropyl; R³ is hydrogen; and R⁴ is hydrogen.

In certain embodiments of a compound of Formula (I) wherein the compound is substantially one diastereomer, the stereochemistry at the carbon to which R² and R³ are bonded is of the S-configuration.

In certain embodiments of a compound of Formula (I) wherein the compound is substantially one diastereomer, the stereochemistry at the carbon atom to which R² and R³ are bonded is of the R-configuration.

In certain embodiments of a compound of Formula (I), the (R)-baclofen prodrug is (3R)-4-{[(1S)-2-methyl-1-(2-methylpropanoyloxy)propoxy]carbonylamino}-3-(4-chlorophenyl)butanoic acid (4), a pharmaceutically acceptable salt thereof, or a pharmaceutically acceptable salt of any of the foregoing.

“Immediate release” refers to formulations or dosage forms that rapidly dissolve in vitro and are intended to be completely dissolved and absorbed in the stomach or upper gastrointestinal tract. Such formulations can release at least 90% of an active compound or precursor thereof within about 15 minutes, within about 30 minutes, within about one hour, or within about two hours following administering the dosage form.

“Minimum adverse concentration” refers to the minimum concentration of a therapeutic compound in, for example, the blood or plasma of a patient, which does not produce an unacceptable adverse drug effect. The unacceptability of an adverse drug effect can be determined, for example, by the patient or the prescribing physician based at least in part on the severity of the adverse drug effect and/or the perceived risk in view of the therapeutic benefits of the compound being administered to the patient. The minimum adverse concentration can also depend, at least in part, on the age, weight and health of the patient being treated, the disease, disorder, or condition being treated, and the judgment of the prescribing physician.

“Minimum therapeutically effective concentration” refers to the minimum concentration of a therapeutic compound in, for example, the blood or plasma of a patient, which produces an intended therapeutic effect.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Pharmaceutical composition” refers to at least one (R)-baclofen prodrug of Formula (I) and at least one pharmaceutically acceptable vehicle, with which the at least one (R)-baclofen prodrug of Formula (I) is administered to a patient.

“Pharmaceutically acceptable” refers to approved or approvable by a regulatory agency of the Federal or a state government or listed in the U.S. Pharmacopeia or other generally recognized pharmacopeia for use in animals, for example, in humans.

“Pharmaceutically acceptable salt” refers to a salt of a compound, which possesses the desired pharmacological activity of the parent compound. Such salts include: acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion, and the like; or coordinates with an organic base such as ethanolamine, diethanolamine, triethanolamine, N-methylglucamine, and the like. In certain embodiments, a pharmaceutically acceptable salt is the hydrochloride salt, and in certain embodiments, the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a (R)-baclofen prodrug of Formula (I) may be administered to a patient and which does not destroy the pharmacological activity thereof, and which is nontoxic when administered in doses sufficient to provide a therapeutically effective amount of the compound.

“Promoiety” refers to a chemical group, i.e. moiety, bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. Cleavage of the promoiety may proceed spontaneously, such as via a hydrolysis reaction, or may be catalyzed or induced by another agent, such as by an enzyme, by light, by acid, or by a change of or exposure to a physical or environmental parameter such as a change of temperature, pH, etc. The agent may be endogenous to the conditions of use, such as an enzyme present in the systemic circulation of a patient to which the prodrug is administered or the acidic conditions of the stomach or the agent may be supplied exogenously. As an example, for a (R)-baclofen prodrug of Formula (I), the promoiety is:

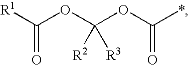

where R¹, R², and R³ are as defined herein, and the drug is (R)-baclofen.

“Patient” refers to a mammal, for example a human.

“Sedation” as used herein refers to minimal sedation and/or moderate sedation (see e.g., American Society of Anesthesiologists, Anesthesiology 2002, 96, 1004-17). Minimal sedation, also referred to as anxiolysis, is a minimally depressed level of consciousness that retains the patient's ability to independently and continuously maintain an airway and respond appropriately to physical stimulation or verbal command that is produced by a pharmacological or non-pharmacological method or combination thereof. Although cognitive function and coordination may be modestly impaired, ventilatory and cardiovascular functions are unaffected. When the intent is minimal sedation in adults, the appropriate dosing is no more than the maximum recommended dose that can be prescribed for unmonitored home use, e.g., a maximum recommended therapeutic dose. Moderate sedation is a drug-induced depression of consciousness during which patients respond purposefully to verbal commands, either alone or accompanied by light tactile stimulation. No intervention is required to maintain a patient's airway. Sedation is a continuum and it is not always possible to predict how an individual patient will respond. A sedative dose can be determined by incremental dosing, administering multiple doses of a drug, such as a prodrug of (R)-baclofen provided by the present disclosure, until a desired effect is reached. A variety of scales can be used to assess sedation including, for example, the Ramsay scale (Ramsay et al., Br Med J 1974, 2, 656-659), and the Observer's Assessment of Alertness/Sedation scale (Chemik et al., J Clin Psychopharmacol 1990, 10, 244-251), and others (see e.g., Sessler, Chest 2004, 126, 1727-1730). Objective measures of sedation include measurement of electroencephalogram parameters such as the Bispectral Index version XP and the Patient State Analyzer (see, e.g., Chisholm et al., Mayo Clin Proc 2006, 81(1), 46-52; and Tonner et al., Best Pract Res Clin Anaesthesiol 2006, 20(1), 191-2000). In certain embodiments, sedation refers to minimal sedation, and in certain embodiments, moderate sedation.

“Solvate” refers to a molecular complex of a compound with one or more solvent molecules in a stoichiometric or non-stoichiometric amount. Such solvent molecules are those commonly used in the pharmaceutical art, which are known to be innocuous to a recipient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a complex where the one or more solvent molecules are water including monohydrates and hemi-hydrates.

“Substantially one diastereomer” refers to a compound containing two or more stereogenic centers such that the diastereomeric excess (d.e.) of the compound is greater than or at least about 90%. The diastereomeric excess is the ration of the percentage of one diastereomer in a mixture to that of another diastereomer. In some embodiments, the diastereomeric excess is, for example, greater than or at least 91%, at least about 92%, at least about 93%, at least about 94%, at least about 95%, at least about 96%, at least about 97%, at least about 98%, or at least about 99%.

“Substituted” refers to a group in which one or more hydrogen atoms are independently replaced with the same or different substituent(s). Examples of substituents include, but are not limited to, -Q, —R60, —O—, —OH, ═O, —OR60, —SR60, —S—, ═S, —NR60R61, ═NR⁶⁰, —CX₃, —CN, —CF₃, —OCN, —SCN, —NO, —NO₂, ═N₂, —N₃, —S(O)₂O⁻, —S(O)₂OH, —S(O)₂R⁶⁰, —OS(O₂)O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹, —C(NR⁶²)NR⁶⁰R⁶¹, —S(O)₂, NR⁶⁰R⁶¹, —NR⁶³S(O)₂R⁶⁰, —NR⁶³C(O)R⁶⁰, and —S(O)R⁶⁰ where each Q is independently a halogen; each R⁶⁰ and R⁶¹ are independently chosen from hydrogen, alkyl, substituted alkyl, alkoxy, substituted alkoxy, cycloalkyl, substituted cycloalkyl, heterocycloalkyl, substituted heterocycloalkyl, aryl, substituted aryl, heteroaryl, substituted heteroaryl, arylalkyl, substituted arylalkyl, heteroarylalkyl, and substituted heteroarylalkyl; or R⁶⁰ and R⁶¹ together with the nitrogen atom to which they are bonded form a ring chosen from a heterocycloalkyl, substituted heterocycloalkyl, heteroaryl, and substituted heteroaryl ring; and R⁶² and R⁶³ are independently chosen from hydrogen, alkyl, substituted alkyl, aryl, substituted aryl, arylalkyl, substituted arylalkyl, cycloalkyl, substituted cycloalkyl, heterocycloalkyl, substituted heterocycloalkyl, heteroaryl, substituted heteroaryl, heteroarylalkyl, and substituted heteroarylalkyl; or R⁶² and R⁶³ together with the atom to which they are bonded form a ring chosen from a heterocycloalkyl, substituted heterocycloalkyl, heteroaryl, and substituted heteroaryl ring. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substitute groups: F, Cl, Br, C₁₋₃ alkyl, substituted C₁₋₃ alkyl, C₁₋₃ alkoxy, substituted C₁₋₃ alkoxy, —S(O)₂NR⁶⁰R⁶¹, —NR⁶⁰R⁶¹, —CF₃, —OCF₃, —CN, —NR⁶⁰S(O)₂R⁶¹, —NR⁶⁰C(O)R⁶¹, C₅₋₁₀ aryl, substituted C₅₋₁₀ aryl, C₅₋₁₀ heteroaryl, substituted C₅₋₁₀ heteroaryl, —C(O)OR¹⁰, —NO₂, —C(O)R⁶⁰, —C(O)NR⁶⁰R⁶¹, —OCHF₂, C₁₋₃ acyl, —SR⁶⁰, —S(O)₂OH, —S(O)₂R⁶⁰, —S(O)R⁶⁰, —C(S)R⁶⁰, —C(O)O, —C(S)OR⁶⁰, —NR⁶⁰C(O)NR⁶¹R⁶², —NR60C(S)NR⁶¹R⁶², and —C(NR⁶⁰)NR⁶¹R⁶²C₃₋₈ cycloalkyl, and substituted C₃₋₈ cycloalkyl, wherein R⁶⁰, R⁶¹, and R⁶² are independently chosen from hydrogen and C₁₋₄ alkyl.

In certain embodiments, each substituent group can independently be chosen from halogen, —NO₂, —OH, —COOH, —NH₂, —CN, —CF₃, —OCF₃, C₁₋₈ alkyl, substituted C₁₋₈ alkyl, C₁₋₈ alkoxy, and substituted C₁₋₈ alkoxy.

“Sustained release” refers to release of a compound such as a drug or prodrug from a dosage form at a rate effective to achieve a therapeutic or prophylactic amount of the drug, in the systemic blood circulation over a prolonged period of time relative to that achieved by oral administration of an immediate release formulation of the compound. In some embodiments, release of the compound occurs over a period of at least about 4 hours, in some embodiments, over a period of at least about 8 hours, in some embodiments over a period of at least about 12 hours, in some embodiments, over a period of at least about 16 hours, in some embodiments, over a period of at least about 20 hours, and in some embodiments, over a period of at least about 24 hours. In some embodiments, a sustained-release dosage form provides a concentration of the therapeutic compound in the blood and/or plasma of a patient, which is greater than a minimum therapeutically effective concentration and less than an adverse concentration for a continuous period of time such as for example for a period of at least about 4 hours, in some embodiments, over a period of at least about 8 hours, in some embodiments, over a period of at least about 12 hours, in some embodiments, over a period of at least about 16 hours, in some embodiments, over a period of at least about 24 hours, and in some embodiments, over a period of at least about 24 hours.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease, disorder, or at least one of the clinical symptoms of a disease or disorder. “Treating” or “treatment” also refers to inhibiting the disease, disorder, or at least one of the clinical symptoms of a disease or disorder, either physically, (e.g., stabilization of a discernible symptom), physiologically, (e.g., stabilization of a physical parameter), or both, and to inhibiting at least one physical parameter which may or may not be discernible to the patient. In certain embodiments, “treating” or “treatment” refers to delaying the onset of the disease or disorder or at least one or more symptoms thereof in a patient which may be exposed to or predisposed to a disease or disorder even though that patient does not yet experience or display symptoms of the disease or disorder.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The “therapeutically effective amount” can vary depending, for example, on the compound, the disease, disorder, and/or symptoms of the disease or disorder, severity of the disease, disorder, and/or symptoms of the disease or disorder, the age, weight, and/or health of the patient to be treated, and the judgment of the prescribing physician. An appropriate therapeutically effective amount in any given instance may be ascertained by those skilled in the art or capable of determination by routine experimentation.

“Therapeutically effective dose” refers to a dose of a drug, prodrug or active metabolite of a prodrug that provides effective treatment of a disease or disorder in a patient. A therapeutically effective dose may vary from compound to compound and from patient to patient, and may depend upon factors such as the condition of the patient and the route of delivery. A therapeutically effective dose may comprise one or more dosage forms. A therapeutically effective dose may be determined in accordance with routine pharmacological procedures known to those skilled in the art.

Reference is now made in detail to embodiments of the present disclosure. The disclosed embodiments are not intended to be limiting of the claims. To the contrary, the claims are intended to cover alternatives, modifications, and equivalents.

### Particles

Certain embodiments provided by the present disclosure provide oral pharmaceutical dosage forms of an (R)-baclofen prodrug, comprising at least two particle populations, wherein at least one of the two particle populations is chosen from (a) and (b): (a) a population of (R)-baclofen prodrug-containing particles, the particles when placed in an aqueous solution releasing the (R)-baclofen prodrug into the solution with a release profile that is independent of the solution pH; and (b) a population of (R)-baclofen prodrug-containing particles, the particles when placed in an aqueous solution releasing the (R)-baclofen prodrug into the solution with a release profile that is dependent on the solution pH; wherein the oral dosage form provides a therapeutically effective concentration of (R)-baclofen in blood and/or plasma of a patient for a continuous period of time after the oral dosage form is orally administered to the patient.

In certain embodiments of oral dosage forms of an (R)-baclofen prodrug comprising at least two particle populations wherein at least one of the two particle populations is chosen from particle population (a) and particle population (b), the dosage form further comprises (c), a population of (R)-baclofen prodrug-containing particles, the population (c) particles when placed in an aqueous solution releasing substantially all of the (R)-baclofen prodrug into the solution within about 1 hour of being placed in the solution.

In certain embodiments, an oral dosage form comprises a combination of particle populations (a) and (b). In certain embodiments, an oral dosage form comprises a combination of a first particle population (a) having a first release profile that is independent of the solution pH, and a second particle population (a) having a second release profile that is independent of the solution pH, wherein the second release profile is different than the first release profile. In certain embodiments, an oral dosage form comprises a combination of a first particle population (b) having a third release profile that is dependent on the solution pH, and a second particle population (b) having a fourth release profile that is dependent on the solution pH, wherein the fourth release profile is different than the third release profile. In certain embodiments, an oral dosage from comprises a combination of particle populations (a) and (c). In certain embodiments, an oral dosage from comprises a combination of particle populations (b) and (c). In certain embodiments, an oral dosage from comprises a combination of particle populations (a), (b), and (c).

In certain embodiments, the (R)-baclofen prodrug is (3R)-4-{[(1S)-2-methyl-1-(2-methylpropanoyloxy)propoxy]carbonylamino}-3-(4-chlorophenyl)butanoic acid, a pharmaceutically acceptable salt thereof, or a pharmaceutically acceptable solvate of any of the foregoing. In certain embodiments, the (3R)-4-{[(1S)-2-methyl-1-(2-methylpropanoyloxy)propoxy]carbonylamino}-3-(4-chlorophenyl)butanoic acid or the pharmaceutically acceptable salt or solvate thereof is in a crystalline form.

Population (a), (b), and (c) particles can comprise cores having a coating containing a (R)-baclofen prodrug. In general, population (c) particles release a (R)-baclofen prodrug from the particles upon contact with gastrointestinal fluid. In addition to a coating comprising a (R)-baclofen prodrug surrounding an inert core, population (a) and (b) particles have a controlled-release coating. The controlled-release coating modifies the release of a (R)-baclofen prodrug from a particle compared to a population (c) particle having the same composition.

Population (c) particles can comprise any form of immediate release (R)-baclofen prodrug. Immediate release particles can comprise uncoated particulates, granules, and/or powders of a (R)-baclofen prodrug, can include particulates, granules, and/or pellets of a (R)-baclofen prodrug coated with a highly soluble immediate release coating such as Opadry® type coating, as are known to those skilled in the art (see, e.g., U.S. Pat. No. 5,098,715), or can comprise inert cores having a coating of a (R)-baclofen prodrug.

Inert cores useful in the practice of the present disclosure can comprise any appropriate type of core material useful in pharmaceutical applications, which can be water insoluble, such as cellulose spheres or silicon dioxide, or can be water soluble such as starch and/or sugar. A core can comprise beads, ion-exchange resin beads, spheroids, spheres, seeds, pellets, granules, or other particulate form. A core can comprise a material such as sugar, starch, sugar and starch, sucrose crystals, extruded and dried spheres comprising excipients such as microcrystalline cellulose and lactose, or an acidic or alkaline buffer crystal such as calcium carbonate, sodium bicarbonate, fumaric acid, tartaric acid which can alter the microenvironment of the drug to facilitate release of the drug. In certain embodiments, a core comprises sugar (Sugar Sphere NF). A core can have any appropriate dimension suitable for oral delivery. For example, a core can have a diameter ranging from about 15 mesh to about 50 mesh, from about 20 mesh to about 25 mesh, or from about 30 mesh to about 35 mesh. In certain embodiments, the diameter of a core can range from about 0.25 mm to about 3 mm, and in certain embodiments, from about 0.5 mm to about 1 mm.

Inert cores can be coated with a formulation comprising a (R)-baclofen prodrug. In certain embodiments, the coating can further include a binding agent to provide adhesion between a core and a (R)-baclofen prodrug. The binding agent can be water-soluble and can include any appropriate binding agent known in the art such as polyvinylpyrrolidone, hydroxyethyl cellulose, hydroxypropyl cellulose, low molecular weight hydroxypropyl methylcellulose (HPMC), polymethacrylate, ethyl cellulose, or combinations of any of the foregoing. In certain embodiments, the binding agent is a polyvinylpyrrolidone polymer such as Povidone USP, EP (Plasdone™ K29/32). In certain embodiments, the amount of binder comprising a coating composition ranges from about 2 wt % to about 10 wt %, and in certain embodiments from about 4 wt % to about 6 wt % based on the total solids weight of the coating composition.

A coating comprising a (R)-baclofen prodrug can be applied to a core by any appropriate method known in the pharmaceutical industry such as fluidized bed coating, rotor granulation, pan coating, or spray coating. A suspension of a (R)-baclofen prodrug and a binder can be formed in a low viscosity solvent such as isopropyl alcohol, ethanol, acetone, water, or mixtures of any of the foregoing. The suspension can then be applied to a core to provide a coating thickness sufficient to provide the desired amount of a (R)-baclofen prodrug. In certain embodiments, such coated cores can be used as population (c) particles.

In certain embodiments, immediate release particles, e.g, population (c) particles, may comprise particles having an active core comprising compound (4). Active cores comprising a (R)-baclofen prodrug of Formula (I) or compound (4) may further comprise any appropriate vehicle, for example, any of those disclosed herein. Cores comprising a (R)-baclofen prodrug of Formula (I) or compound (4) may be granules formed by granulation methods known to those skilled in the art. Cores comprising a (R)-baclofen prodrug of Formula (I) or compound (4) may be coated with a coating that may or may not comprise a (R)-baclofen prodrug of Formula (I) or compound (4).

In certain embodiments, one or more additional coatings can be applied to particles having a coating of a (R)-baclofen prodrug. The purpose of the one or more additional coatings can be for physical protection, and/or to facilitate further processing of the particles. These sealant or barrier coatings are not intended to modify or affect the release of a (R)-baclofen prodrug from the oral dosage form in the gastrointestinal tract. These additional coatings can also be applied to particles, e.g., immediate-release particles, comprising a (R)-baclofen prodrug by methods known to those skilled in the art and as disclosed herein. Examples of materials useful in coatings for physical protection include permeable or soluble materials such as hydroxypropyl methylcellulose, hydroxypropyl cellulose, hydroxypropyl ethylcellulose, and xanthan gum. Examples of materials useful in coatings to facilitate further processing include talc, colloidal silica, polyvinyl alcohol, titanium dioxide, micronized silica, fumed silica, glycerol monostearate, magnesium trisilicate, magnesium stearate, and combinations of any of the foregoing.

In certain embodiments, one or more additional coatings that impart desired release properties can be applied to a core having a coating of a (R)-baclofen prodrug. Such particles are referred to herein as controlled-release particles. Controlled release particles have a controlled-release coating surrounding a coating of a (R)-baclofen prodrug on an inert core. A controlled-release coating modifies or controls the release of a (R)-baclofen prodrug from a controlled-release particle in the gastrointestinal tract. Controlled-release coating materials include bioerodible, gradually hydrolysable, gradually water-soluble, enzymatically degradable polymers, and/or enteric polymers. Enteric polymers become soluble in the higher pH environment of the lower gastrointestinal tract or slowly erode as the polymer passes through the gastrointestinal tract, while enzymatically degradable polymers are degraded by bacterial enzymes present in the lower gastrointestinal tract, for example, in the colon. For example, in certain embodiments where a controlled-release particle having a coating of a (R)-baclofen prodrug is further coated with a pH-dependent release polymer, which is insoluble in the acid environment of the stomach, insoluble in the environment of the small intestines, and soluble in the conditions within the lower small intestine or upper large intestine (e.g., above pH 7.0) release of a (R)-baclofen prodrug from the particle can be minimized or prevented in the upper part of the gastrointestinal tract.

Examples of coating materials for effecting controlled or modified release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropyl methyl cellulose phthalate, methylcellulose, ethyl cellulose, cellulose acetate, cellulose acetate phthalate, cellulose acetate trimellitate, and carboxymethylcellulose sodium; acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate co-polymers, ammonio methacrylate copolymers, and methacrylic resins commercially available under the tradename Eudragt™ including Eudragit™ L, Eudragit™ S, Eudragit™ E, Eudragit™ RL, and Eudragit™RS; vinyl polymers and copolymers such as polyvinylpyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylase, and guar gum; and shellac. Combinations of any of the foregoing polymers may also be used to form a controlled-release coating. In certain embodiments, more than one controlled release coating can be used to modify the release properties of a (R)-baclofen pro drug from a particle.

In certain embodiments, a controlled-release coating can provide pH-dependent release of a (R)-baclofen prodrug from a particle having a coating of a (R)-baclofen prodrug in the gastrointestinal tract. pH-dependent release particles are also referred to herein as population (b) particles. A pH-dependent coating is designed to release a (R)-baclofen prodrug from a particle in a desired area or areas of the gastrointestinal tract such as the small intestine and/or colon depending on the pH of the gastrointestinal fluid in the desired area of the gastrointestinal tract. Examples of pH-dependent coating materials useful in certain embodiments provided by the present disclosure include shellac, cellulose acetate phthalate, polyvinyl acetate phthalate, hydroxypropyl methylcellulose phthalate, and methacrylic acid ester copolymers, zein, and the like. In certain embodiments, a pH-dependent coating can be a copolymer synthesized from diethylaminoethyl methacrylate and other neutral methacrylic esters, also known as methacrylic acid copolymers or polymer methacrylates, commercially available as Eudragit™ (Rohm Pharma). There are several different types of Eudragit™ polymers useful for imparting pH-dependent release properties. For example, Eudragit™ E is a methacrylic copolymer that swells and dissolves in acidic media. Eudragit™ L is a methacrylic acid copolymer that does not swell at about pH<5.7 and is soluble at about pH>6. Eudragit™ S does not swell at about pH<6.5 and is soluble at about pH>7. Eudragit™ S and Eudragit™ L can be used as single components in a controlled-release coating or in a combination in any ratio to achieve desired release properties. By using a combination of copolymers, a controlled release coating can exhibit a solubility at a pH between the pHs at which, for example, Eudragit™ S and Eudragit™ L are separately soluble.

In certain embodiments, a controlled-release coating can provide pH-independent release of a (R)-baclofen prodrug from a particle having a coating of a (R)-baclofen prodrug in the gastrointestinal tract. pH-independent release particles are also referred to herein as population (a) particles. A pH-independent coating is designed to release a (R)-baclofen prodrug from a controlled-release particle at a rate independent of the pH. Ammonioalkyl methacrylate copolymers such as Eudragit™ RS and Eudragit™ RL are examples of useful pH-independent polymers. Eudragit™ RL and Eudragit™ RS are acrylic resins comprising copolymers of acrylic and methacrylic acid esters with a low content of quaternary ammonium groups. The ammonium groups are present as salts and impart permeability to the lacquer films. Eudragit™ RL and Eudragit™ RS swell in water and digestive juices, in a pH-independent manner. In the swollen state coatings formed therefrom are permeable to water and release or dissolve active compounds. Eudragit™ RL and Eudragit™ RS can be used alone in a pH-independent coating, combined together, combined with other ammonioalkyl methacrylate copolymers, or other methacrylic acid copolymers to achieve a desired release property.

In certain embodiments, a controlled-release coating can comprise a pharmaceutically acceptable acrylic polymer including, for example, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate co-polymers, ammonioalkyl methacrylate copolymers, and combinations of any of the foregoing.

A coating comprising a (R)-baclofen prodrug and/or other coating used to form immediate- or controlled-release particles provided by the present disclosure such as a sealant coating, a barrier coating, a pH-dependent release coating, and a pH-independent release coating can also comprise one or more pharmaceutically acceptable excipients such as, for example, surfactants, lubricants, diluents, plasticizers, anti-adherents, glidants, buffers, disintegrants, fillers, wetting agents, emulsifying agents, pH buffering agents, pH-modifying agents, stabilizing agents, chelating agents, binders, thickening agents, coloring agents, and combinations of any of the foregoing.

Examples of surfactants useful in pharmaceutically acceptable coatings provided by the present disclosure include pharmaceutically acceptable anionic surfactants, cationic surfactants, amphoteric (amphiphatic/ampophilic) surfactants, non-ionic surfactants, polyethyleneglycol esters or ethers, and combinations of any of the foregoing. Examples of useful pharmaceutically acceptable anionic surfactants include monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates such as sodium lauryl sulfate, ethoxylated alkyl sulfates, ester linked sulfonates such as docusate sodium and dioctyl sodium succinate, alpha olefin sulfonates, or phosphated ethoxylated alcohols. Examples of pharmaceutically acceptable cationic surfactants useful in coatings provided by the present disclosure include monoalkyl quaternary ammonium slats, dialkyl quaternary ammonium compounds, amidoamines, and aminimides. Examples of useful pharmaceutically acceptable amphoteric surfactants include N-substituted alkyl amides, N-alkyl betaines, sulfobetaines, and N-alkyl-6-aminopropionates. Examples of pharmaceutically acceptable polyethyleneglycol esters or ethers useful in coatings provided by the present disclosure include polyethoxylated castor oil, polyethoxylated hydrogenated castor oil, or hydrogenated castor oil.

Lubricants and anti-static agents may be included in a pharmaceutically acceptable coating to aid in processing. Examples of lubricants useful in coatings provided by the present disclosure include calcium stearate, glycerol behenate, glyceryl monostearate, magnesium stearate, mineral oil, polyethylene glycol, sodium stearyl fumarate, sodium lauryl sulfate, stearic acid, talc, vegetable oil, zinc stearate, and combinations of any of the foregoing. In certain embodiments, the lubricant is glyceryl monostearate. In certain embodiments, coatings may comprise an amount of lubricant ranging from about 1 wt % to about 15 wt % based on the total solids weight of the coating.

Plasticizers may be included in pharmaceutically acceptable coatings to improve the physical properties of the cured coating. For example, plasticizers may increase the flexibility or elasticity of a coating comprising a film-forming material having a relatively high glass transition temperature such as ethyl cellulose. Examples of plasticizers useful in coatings provided by the present disclosure include adipates, azelate, benzoates, citrates, isoebucates, phthalates, sebacates, stearates, glycols, polyethylene glycol, propylene glycol, triacetin, dimethyl phthalate, diethyl phthalate, dibutyl phthalate, dibutyl sebacate, triethyl citrate, tributyl citrate, triethyl acetyl citrate, acetyltributylcitrate, glyceryl triacetate, castor oil, acetylated monoglycerides, and combinations of any of the foregoing.

Glidants may be included in pharmaceutically acceptable coatings to reduce sticking effects during processing, film formation, and/or drying. Examples of glidants useful in coatings provided by the present disclosure include talc, magnesium stearate, glycerol monostearate, colloidal silicon dioxide, precipitated silicon dioxide, and combinations of any of the foregoing.

Examples of pH-buffering agents useful in coatings provided by the present disclosure include citric acid, sodium citrate, fumaric acid, sodium fumarate, and combinations of any of the foregoing.

pH modifying agents can create a microenvironment around released (R)-baclofen prodrug or (R)-baclofen metabolite when exposed to aqueous fluids. For example, pH-modifying agents may drive the prodrug or the (R)-baclofen metabolite to its net neutral form, thereby enhancing its absorption through the intestinal epithelia. Examples of pharmaceutically acceptable alkaline pH modifying agents include, for example, L-lysine, L-arginine, sodium citrate, and magnesium hydroxide. Examples of pharmaceutically acceptable acidic pH modifying agents include, for example, fumaric acid, citric acid, tartaric acid, malic acid, maleic acid, and succinic acid.

Examples of stabilizers useful in coatings provided by the present disclosure include anti-oxidants such as 3,5-di-tert-butyl-4-hydroxytoluene (BHA), 3-(or 2)-tert-butyl-4-hydroxyanisole (BHT), ascorbic acids, tocopherols, and the like.

Binders may be included in coating compositions to hold the components of a coating together. Examples of binders useful in coatings provided by the present disclosure include, for example, polyvinylpyrrolidone, hydroxypropyl cellulose, hydroxypropyl methylcellulose, methylcellulose, hydroxyethyl cellulose, sugars, dextran, cornstarch, and combinations of any of the foregoing. In certain embodiments, the binder is polyvinylpyrrolidone such as Plasdone® K29/32 Plasdone (ISP Technologies, Wayne, N.J.).

Anti-foaming agents may also be included in pharmaceutically acceptable coatings. Examples of anti-foaming agents useful in coatings provided by the present disclosure include silicone and simethicone.

Examples of pigments useful in coatings provided by the present disclosure include titanium dioxide, food color lakes, and iron oxides.

Controlled release coatings provided by the present disclosure may also comprise erosion-promoting agents such as starch and gums, materials useful for making microporous lamina in the use environment such as polycarbonates characterized by linear polyesters of carbonic acid in which carbonate groups reoccur in the polymer chain, and/or semi-permeable polymers such as hydroxypropylmethyl cellulose, lactose, and metal stearates.

Release of a prodrug of (R)-baclofen from controlled release particles may further be influenced, for example, adjusted to a desired release rate, by providing one or more pores or passageways through one or more coatings. Release-modifying materials, which may be incorporated into controlled release coatings, and which function as pore-formers may be organic or inorganic, include materials that can be dissolved, extracted, or leached from the coating in the environment of use, e.g., the gastrointestinal tract or in a certain region or regions of the gastrointestinal tract, such as hydrophilic materials, for example, hydroxypropylmethyl cellulose.

The combination of all solid components of the polymeric material, including co-polymers, fillers, plasticizers, and optional excipients and processing aids, can provide an about 10% to about 450% weight gain to the cores. In certain embodiments, the weight gain is about 30 to about 160%.

Any of the controlled-release materials can be combined with one or more other controlled release materials to provide a controlled-release coating, or can be provided as separate coatings, eac coating comprising one or more controlled-release materials applied to a core comprising a coating of a (R)-baclofen prodrug.

### Dosage Forms

Certain embodiments provided by the present disclosure provide oral dosage forms containing at least one population of particles disclosed herein, in the form of a liquid, liquid suspension, gel, capsule, tablet, chewable tablet, crushable tablet, rapidly dissolving tablet, or sachet or capsule for reconstitution. In certain embodiments, a dosage form can be a capsule, a tablet, or a liquid suspension. A dosage form can be of any shape suitable for oral administration of a drug, such as spheroidal, cube-shaped oval, or ellipsoidal. A dosage form can be prepared from the particles in a manner known in the art and can further include additional pharmaceutically acceptable vehicles, as appropriate.

In certain embodiments, one or more populations of controlled-release particles and optional population of immediate release particles can be placed in a hard or soft gelatin capsule in an amount sufficient to provide a therapeutically effective concentration of (R)-baclofen when orally ingested.

In certain embodiments, one or more populations of controlled release particles and optional population of immediate-release particles can be compressed into a tablet using tableting equipment and known techniques. Techniques and compositions for making tablets (compressed and molded) capsules (hard and soft gelatin), and pills are also described in, for example, “The Science and Practice of Pharmacy,” 21^{st} Ed., Lippincott Williams & Wilkins, 2005, Chapter 45. Tablet dosage forms can be prepared by various conventional mixing, commination and fabrication techniques readily apparent to those skilled in the chemistry of drug formulations. Examples of such techniques include direct compression using appropriate punches and dies, the punches and dies are fitted to a suitable rotary tableting press; injection or compression molding using suitable molds fitted to a compression unit; granulation followed by compression; and extrusion in the form of a paste, into a mold or to an extrudate to be cut into lengths. Excipients useful in tablet formulations include, for example, an inert diluent such as lactose, granulating and disintegrating agents, such as cornstarch, binding agents, such as starch, and lubricating agents, such as magnesium stearate. A tablet can comprise a unit dose of a (R)-baclofen prodrug, or in the case of a mini-tablet, which comprises less than a unit dose, mini-tablets can be filled into capsules to provide a unit dose. In certain embodiments, a tablet can be a multilayer tablet in which different layers contain different particle populations and/or different excipients that affect the release properties of a (R)-baclofen prodrug from each tablet layer. Tablets can be disintegrating tablets, fast dissolving tablets, effervescent tablets, fast melt tablets, chewable tablets, crushable tablets, and/or mini-tablets.

Disintegrants can be included in a tablet formulation to cause a tablet to break apart, for example, by expansion of a disintegrants when exposed to water. Examples of useful disintegrants include water swellable substances such as low-substituted hydroxypropyl cellulose, cross-linked sodium carboxymethylcellulose (sodium croscarmellose), sodium starch glycolate, sodium carboxymethylcellulose, sodium carboxymethyl starch, ion-exchange resins, microcrystalline cellulose, starches and pregelatinized starch, formalin-casein, alginic acid, certain complex silicates, and combinations of any of the foregoing.

Fillers can be included in dosage forms provided by the present disclosure. A filler can be a water insoluble filler, water soluble filler, or combinations of any of the foregoing. Examples of useful water insoluble fillers include silicon dioxide, titanium dioxide, talc, alumina, starch, kaolin, polacrilin potassium, powdered cellulose, microcrystalline cellulose, fumed silica, glyceryl monostearate, magnesium stearate, calcium stearate, colloidal silica, micronized silica, magnesium trisilicate, gypsum, and combinations of any of the foregoing. Examples of water-soluble fillers include water soluble sugars and sugar alcohols, such as lactose, glucose, fructose, sucrose, mannose, dextrose, galactose, the corresponding sugar alcohols and other sugar alcohols, such as mannitol, sorbitol, xylitol, and combinations of any of the foregoing.

In certain embodiments, a dosage form can comprise a suspension in which one or more populations of particles comprising a (R)-baclofen prodrug are dispersed in a pharmaceutically acceptable solvent formulation. A solvent formulation can include water, ethanol, flavorings, colorings, or combinations of any of the foregoing. Liquid oral dosage forms can include aqueous and non-aqueous solutions, emulsions, suspensions, and solutions and/or suspensions reconstituted from non-effervescent granules, containing suitable solvents, emulsifying agents, suspending agents, diluents, sweeteners, coloring agents, and flavoring agents, preservatives, and combinations of any of the foregoing. The solvent of an aqueous-based orally acceptable pharmaceutical carrier is entirely or predominantly water and can include a suspending agent. Examples of carriers include aqueous solutions, syrups, elixirs, dispersions, suspensions, emulsions such as oil-in-water emulsions, and microemulsions. Examples of suspending agents include microcrystalline cellulose/sodium carboxymethyl cellulose, guar gum, and the like. Co-solvents useful to solubilize and incorporate water-insoluble ingredients into a suspension include propylene glycol, glycerin, sorbitol solution, and the like. In addition, a liquid formulation can include excipients such as wetting agents, emulsifying and suspension agents, sweetening, flavoring, coloring, perfuming, and preserving agents. Examples of suspension agents include ethoxylated isostearyl alcohols, polyoxyethylene sorbitol and sorbitan esters, microcrystalline cellulose, aluminum metahydroxide, bentonite, agar-agar, tragacanth, and combinations of any of the foregoing.

In certain embodiments, controlled-release particles and optional immediate-release particles can be incorporated into gels such as ion-exchange resin containing gel compositions.

In certain embodiments, controlled-release particles and optional immediate-release particles can be provided in a sachet, capsule, or other suitable packaging material as a unit dose in which the contents can be reconstituted at the time of use into a suitable vehicle such as water. The dose-containing package can further include excipients to facilitate the dispersion of the particles in water.

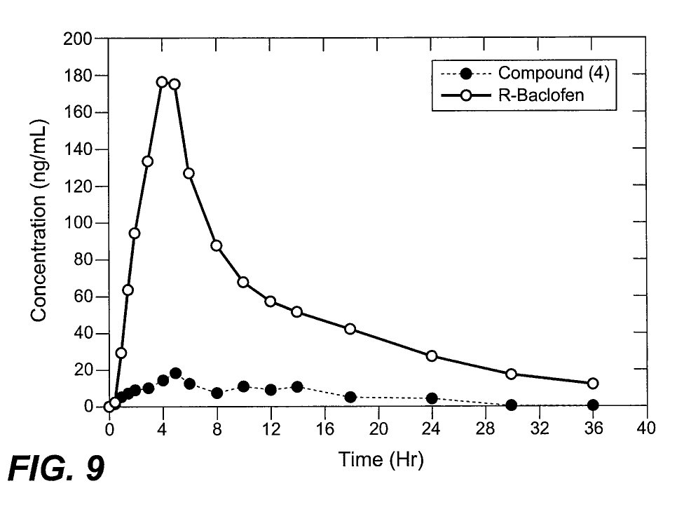

For dosage forms comprising more than one population of particles, the relative quantity of each population of particles can depend on the release profile of a (R)-baclofen prodrug in the gastrointestinal tract intended to be achieved. In certain embodiments, it can be desirable that a dosage form comprising (R)-baclofen prodrug provides a sustained therapeutically effective concentration of (R)-baclofen in the blood and/or plasma of a patient for a continuous period of time after the dosage form is orally administered to the patient. In certain embodiments, a therapeutically effective concentration of (R)-baclofen in the blood and/or plasma of a patient ranges from about 1 ng/mL to about 400 ng/mL, and in certain embodiments, from about 10 ng/mL to about 200 ng/mL. In certain embodiments, a dosage form can provide a blood or plasma concentration profile of (R)-baclofen substantially as shown in any one of FIGS. 6 and 9 following oral administration to a patient. In certain embodiments, a dosage from can provide a blood or plasma concentration profile of (R)-baclofen that is bioequivalent to a profile shown in FIGS. 6 and 9. Achieving such sustained release profiles will depend, at least in part, on the pharmacokinetics of a (R)-baclofen prodrug and/or (R)-baclofen, as well as the release characteristics of compound (4) from the dosage form. The pharmacokinetics of a (R)-baclofen prodrug refers to the absorption, distribution, metabolism, and excretion (ADME) of a (R)-baclofen prodrug in a patient to which a dosage form comprising a (R)-baclofen prodrug is administered.

In certain embodiments, an oral sustained release dosage form containing a (R)-baclofen pro drug comprises a mass equivalent of (R)-baclofen or a pharmaceutically acceptable salt or solvate thereof ranging from about 0.1 mg to about 100 mg, in certain embodiments from about 0.5 mg to about 80 mg, and in certain embodiments from about 2 mg to about 40 mg.

In certain embodiments, a dosage form is substantially free of lactam side products formed by intramolecular cyclization of a (R)-baclofen prodrug and/or (R)-baclofen. A dosage form can be stable to extended storage, such as for example, greater than one year, without substantial lactam formation such as less than 0.5% lactam by weight, less than 0.2% lactam by weight, or less than 0.1% lactam by weight.

In certain embodiments, methods of preparing an oral dosage form of an (R)-baclofen prodrug, such as a (R)-baclofen prodrug, or a pharmaceutically acceptable salt or solvate thereof wherein at least one of the particle populations is chosen from (a) and (b), comprising coating cores with a formulation comprising the (R)-baclofen prodrug to form at least one particle (c) population; coating particles of at least one particle (c) population with a pH-independent release coating to form at least one particle (a) population, coating particles of at least one particle (c) population with a pH-dependent release coating to form at least one particle (b) population; and combining at least two particle populations wherein at least one of the particle populations is chosen from (a) and (b) in a pharmaceutically acceptable oral delivery vehicle to provide the oral dosage form. In certain embodiments, the combination of at least two particle populations is chosen from particle populations (a) and (c), particle populations (a) and (b), particle populations ((b) and (c), and particle populations (a), (b), and (c).

Controlled release capsules comprise a plurality of controlled release particles, each of the controlled release particles comprising immediate release particles, which comprise a core and a coating comprising compound (4) and a binder. Immediate release particles release compound (4) upon contact with gastrointestinal fluid more rapidly than controlled release particles and in a manner that does not affect the rate of absorption of compound (4) from the gastrointestinal tract.

Cores may be inert or active. Active cores comprise compound (4) and optionally additional components such a binder.

In embodiments in which cores are inert, inert cores are coated with an immediate release coating comprising compound (4) to form immediate release particles. In embodiments in which cores are active, e.g., contain compound (4), cores comprising compound (4) may be coated with an immediate release coating to provide immediate release particles, or cores comprising compound (4), with or without a coating, may be used as immediate release particles. In certain embodiments, immediate release particles may comprise uncoated granules or powders of compound (4), may comprise inert cores having a coating of compound (4), or may include granules or pellets of compound (4) coated with a highly soluble immediate release coating.

Inert cores may comprise any appropriate type of core material useful in pharmaceutical applications, and which may be water insoluble, such as cellulose spheres or silicon dioxide, or may be water soluble such as starch and/or sugar. Cores may comprise beads, ion-exchange resin beads, spheroids, spheres, seeds, pellets, granules, or other particulate form. Cores may comprise a material such as sugar, starch, sugar and starch, sucrose crystals, extruded and dried spheres comprising excipients such as microcrystalline cellulose and lactose, or an acidic or alkaline buffer crystal such as calcium carbonate, sodium bicarbonate, fumaric acid, tartaric acid, or combinations of any of the foregoing, which may alter the microenvironment of the compound (4) to facilitate release of the compound (4) and/or impact the chemical stability of compound (4). In certain embodiments, inert cores comprise sugar (Sugar Sphere NF). Inert cores may have any appropriate dimension suitable for oral delivery. For example, inert cores may have a diameter ranging from about 15 mesh to about 50 mesh, from about 20 mesh to about 25 mesh, or from about 30 mesh to about 35 mesh. In certain embodiments, inert cores may have a diameter ranging from about 0.25 mm to about 3 mm, and in certain embodiments, from about 0.5 mm to about 1 mm.

To form immediate release particles, inert cores may be coated with a formulation comprising compound (4) that provides for immediate release of compound (4). Coatings may further comprise a binding agent to provide adhesion between cores and compound (4). Binding agents may be water-soluble and may include any appropriate binding agent k such as polyvinylpyrrolidone, hydroxyethyl cellulose, hydroxypropyl cellulose, low molecular weight hydroxypropylmethyl cellulose (HPMC), polymethacrylate, ethyl cellulose, or combinations of any of the foregoing. In certain embodiments, a binding agent is a polyvinylpyrrolidone polymer such as Plasdone® K29/32 Povidone USP/NF. Plasdone K29-32 is a linear homopolymer of vinyl pyrrolidone having a K-value from about 29 to about 32, a viscosity in 5% aqueous solution of about 2.5 cp at 25° C., a nominal molecular weight of 58×103, and a glass transition temperature, T_{g}, of 164° C. In certain embodiments, coating compositions comprise an amount of binder ranging from about 2 wt % to about 10 wt %, and in certain embodiments from about 4 wt % to about 6 wt % based on the total solids weight of the coating composition.

In certain embodiments, controlled-release coatings may provide pH-independent release of compound (4). pH-independent release coatings release compound (4) from controlled release particles at a rate independent of the pH of the fluid in which the particles are immersed. Ammonioalkyl methacrylate copolymers such as Eudragit® RS and Eudragit® RL are examples of useful pH-independent release polymers. Eudragit® RL and Eudragit® RS are acrylic resins comprising copolymers of acrylic and methacrylic acid esters with a low content of quaternary ammonium groups. The ammonium groups are present as salts and impart permeability to the lacquer films formed by the cured resins. Eudragit® RL and Eudragit® RS swell in water and gastrointestinal fluids in a pH-independent manner. In the swollen state the coatings are permeable to water and release or dissolve active compounds. Eudragit® RL and Eudragit®RS may be used alone in pH-independent release coatings, combined together, combined with other ammonioalkyl methacrylate copolymers, or combined with other methacrylic acid copolymers to achieve a desired release property. In certain embodiments, pH independent release polymers are ammonioalkyl methacrylate copolymers such as Eudragit® RL 100.

Controlled release particles comprising compound (4) and a release rate modifying coating may be incorporated into a number of oral dosage forms including capsules, tablets, and liquid suspensions. In certain embodiments, controlled release particles may be placed in a hard or soft gelatin capsule in an amount sufficient to provide a therapeutically effective concentration of (R)-baclofen in the blood of a patient when orally ingested. Capsules comprising controlled release particles are referred to herein as controlled release capsules. In certain embodiments, controlled release capsules comprise a therapeutically effective amount of compound (4) such as for example, form about 1 mg to about 100 mg of compound (4). In certain embodiments, controlled release capsules may comprise less than a therapeutically effective amount of compound (4), in which case multiple capsules may be administered simultaneously or over a period of time to provide a therapeutically effective amount of compound (4).

### Release Characteristics of Particles and Dosage Forms

When administered orally to a patient, i.e., by a patient swallowing a dosage form provided by the present disclosure, a dosage form can provide a sustained therapeutically effective concentration of (R)-baclofen in the blood and/or plasma of the patient during a continuous period of time. In certain embodiments, an oral dosage form can provide a concentration of (R)-baclofen in the blood and/or plasma of a patient that is greater than a minimum therapeutically effective concentration, and less than a minimum adverse concentration of (R)-baclofen in the blood and/or plasma of the patient. In certain embodiments, oral dosage forms provided by the present disclosure provide a therapeutically effective concentration (R)-baclofen in the blood and/or plasma of a patient for a continuous period of time without exceeding the minimum adverse concentration of (R)-baclofen. In certain embodiments, the concentration of (R)-baclofen in the blood and/or plasma of a patient does not exceed a minimum adverse concentration at any time after the dosage form is orally administered to the patient. Oral dosage forms provided by the present disclosure can provide a therapeutically effective concentration of (R)-baclofen in the blood and/or plasma of a patient for a continuous period of time while reducing or eliminating adverse drug effects associated with the high blood and/or plasma concentrations of (R)-baclofen, e.g. at concentrations above the minimum adverse concentration, observed following oral dosing of (R)-baclofen dosage forms. The high oral bioavailability of (R)-baclofen achievable by dosage forms comprising a (R)-baclofen prodrug provided by the present disclosure can facilitate the use of lower mass equivalents of (R)-baclofen in a dose to achieve a sustained therapeutically effective concentration of (R)-baclofen in the blood and/or plasma of a patient compared to the amount of (R)-baclofen in an oral dosage form comprising (R)-baclofen.

A (R)-baclofen prodrug such as a compound of Formula (I) and compound (4) can exhibit enhanced oral bioavailability as (R)-baclofen compared to the oral bioavailability of an equivalent amount of (R)-baclofen when administered in an equivalent dosage form. The enhanced oral bioavailability of a (R)-baclofen prodrug is believed to be due the efficient absorption of a (R)-baclofen prodrug throughout the gastrointestinal tract, including the colon, via passive and/or active transport mechanisms.

The human gastrointestinal tract includes the small intestine and the large intestine. The human small intestine is a convoluted tube about twenty feet in length between the stomach and large intestine. The small intestine is subdivided into the duodenum, the jejunum, and the ileum. The large intestine is about 5 feet in length and runs from the ileum to the anus. The large intestine is divided into the caecum, colon, and the rectum. The colon is divided into four parts including the ascending, traverse, descending, and the sigmoid flexure. In general, an orally ingested compound resides about 1 to 6 hours in the stomach, about 2 to 7 hours in the small intestine, and about 8 to 18 hours in the colon. Thus, the greatest period of time for sustained release of a compound occurs when the compound is passing through the colon.

(R)-Baclofen prodrugs such as compound (4) exhibit enhanced absorption during the extended period of time that the compound passes through the gastrointestinal tract and are absorbed by active transport, passive transport, or a combination of active and passive transport mechanisms. Increased absorption and in particular colonic absorption of a (R)-baclofen prodrug can result in increased systemic bioavailability of a (R)-baclofen prodrug over an extended period of time. Systemic bioavailability refers to the rate and extent of systemic exposure to a drug or an active metabolite thereof, e.g. (R)-baclofen, as reflected in the integrated blood or plasma concentration over a period of time, also referred to as “area under the curve” (AUC). (R)-Baclofen prodrugs such as compound (4) is believed to be capable of absorption over a significant length of the gastrointestinal tract, including the large intestine, and in particular the colon.

Dosage forms, upon releasing a (R)-baclofen prodrug, can provide (R)-baclofen upon in vivo administration to a patient. The promoiety of a (R)-baclofen prodrug can be cleaved either chemically and/or enzymatically. One or more enzymes, such as esterases, present in the intestinal lumen, intestinal tissue, blood, liver, brain, and/or any other suitable tissue of a mammal can enzymatically cleave the promoiety of a (R)-baclofen prodrug. If the promoiety is cleaved after absorption by the gastrointestinal tract, a (R)-baclofen prodrug can be absorbed into the systemic circulation from the large intestine. In certain embodiments, the promoiety of a (R)-baclofen prodrug can be cleaved after absorption by the gastrointestinal tract. In certain embodiments, the promoiety can be cleaved in the gastrointestinal tract and (R)-baclofen can be absorbed into the systemic circulation from the gastrointestinal tract, including the large intestine. In certain embodiments, a (R)-baclofen prodrug can be absorbed into the systemic circulation from the gastrointestinal tract, and the promoiety can be cleaved in the systemic circulation, after absorption of a (R)-baclofen prodrug from the gastrointestinal tract.

Sustained release dosage forms provided by the present disclosure are capable of providing a sustained therapeutically effective concentration of (R)-baclofen in the blood of a patient following oral administration. For example, dosage forms may provide a sustained therapeutically effective concentration of (R)-baclofen in the blood of a patient during a continuous time period chosen from at least about 4 hours, at least about 8 hours, at least about 12 hours, at least about 16 hours, at least about 20 hours, or at least about 24 hours, after oral administration to a patient. In certain embodiments, the concentration of (R)-baclofen in the blood of a patient will not exceed a minimum adverse concentration at any time after the dosage form is orally administered to the patient, e.g., will not reach a concentration that causes adverse events in the particular patient. In certain embodiments, a minimum therapeutically effective blood (R)-baclofen concentration will be about 2 ng/mL, about 5 ng/mL, about 10 ng/mL, about 20 ng/mL, about 30 ng/mL, about 40 ng/mL, about 50 ng/mL, or about 60 ng/mL. In certain embodiments, a therapeutically effective blood concentration of (R)-baclofen for treating is from about 1 ng/mL to less than about 400 ng/mL, and in certain embodiments from about 10 ng/mL to less than about 200 ng/mL.

In certain embodiments, it may be desirable that the blood concentration of (R)-baclofen be maintained at a level between a concentration that causes moderate sedation and/or impaired motor coordination in the patient and a minimum therapeutically effective concentration for treating a disease in a patient for a continuous period of time. The blood concentration of (R)-baclofen that causes moderate sedation or impaired motor coordination in a patient can vary depending on the individual patient.

In certain embodiments, a therapeutically effective dose of a prodrug of a GABA_{B} agonist provides a blood concentration of the corresponding GABA_{B} agonist that is therapeutically effective for treating a disease in a patient, and that is less than a concentration effective in causing moderate sedation and/or impaired motor coordination in the patient, for example, less than about 400 ng/mL, less than about 200 ng/mL, or less than about 100 ng/mL. In certain embodiments, methods provided by the present disclosure provide a blood (R)-baclofen concentration that, following oral administration to a patient, does not produce sedation and/or impaired motor coordination in the patient. In certain embodiments, methods provided by the present disclosure provide a blood (R)-baclofen concentration that, following oral administration to a patient, produces moderate sedation in a patient.

The pharmacokinetic profile of the blood (R)-baclofen concentration can be characterized by a lower Cₘₐₓ/C₁₂ ratio, and a lower Cₘₐₓ/dose, compared to immediate release and sustained release oral formulations comprising (R)-baclofen that provide a similar (R)-baclofen blood AUC_{inf}.

In certain embodiments, an oral dosage form comprising a plurality of pH-independent release particles comprising a (R)-baclofen prodrug of Formula (I), following oral administration to a human patient provides a blood (R)-3-amino-3-(4-chlorophenyl)butanoic acid concentration characterized by: a Cₘₐₓ/C₁₂ ratio ranging from about 1 to about 6; a Cₘₐₓ/dose ratio ranging from about 1.25 (10⁶·mL)⁻¹ to about 3.25 (10⁶·mL)⁻¹; and an AUC_{inf}/dose ratio ranging from about 13 (hr/10⁶·mL) to about 33 (hr/10⁶·mL).

In certain embodiments, an oral dosage form comprising a plurality of pH-independent release particles comprising a (R)-baclofen prodrug of Formula (I), following oral administration to the human patient provides a blood (R)-3-amino-3-(4-chlorophenyl)butanoic acid concentration characterized by: a Cₘₐₓ/C₁₂ ratio ranging from about 2.3 to about 4.3; a Cₘₐₓ/dose ratio ranging from about 1.75 (106·mL)⁻¹ to about 2.75 (10·mL)⁻¹; and an AUC_{inf}/dose ratio ranging from about 18 (hr/10⁶·mL) to about 28 (hr/10⁶·mL).

In certain embodiments, an oral dosage form comprising a plurality of pH-independent release particles comprising a (R)-baclofen prodrug of Formula (I), following oral administration to the human patient provides a blood (R)-3-amino-3-(4-chlorophenyl)butanoic acid concentration characterized by: a Cₘₐₓ/C₁₂ ratio ranging from about 2.8 to about 3.8; a Cₘₐₓ/dose ratio ranging from about 2.0 (10⁶·mL)⁻¹ to about 2.5 (10⁶·mL)⁻¹; and an AUC_{inf}/dose ratio ranging from about 18 (hr/10⁶·mL) to about 28 (hr/10⁶·mL).

In certain embodiments, an oral dosage form comprising a plurality of pH-independent release particles comprising a (R)-baclofen prodrug of Formula (I), following oral administration to the human patient provides an oral bioavailability of (R)-3-amino-3-(4-chlorophenyl)butanoic acid ranging from about 20% to about 72%.

In certain embodiments, an oral dosage form comprising a plurality of pH-independent release particles comprising a (R)-baclofen prodrug of Formula (I), when administered orally to a patient provides a (R)-baclofen plasma concentration profile substantially as shown in FIG. 6.

In certain embodiments, an oral dosage form comprising a plurality of pH-independent release particles comprising a (R)-baclofen prodrug of Formula (I), when administered orally to a patient provides a (R)-baclofen plasma concentration profile that is bioequivalent to the profile shown in FIG. 6.

In certain embodiments, an oral dosage form comprising a plurality of pH-independent release particles comprising a (R)-baclofen prodrug of Formula (I), the (R)-baclofen prodrug is 3R)-4-{[(1S)-2-methyl-1-(2-methylpropanoyloxy)propoxy]carbonylamino}-3-(4-chlorophenyl)butanoic acid, a pharmaceutically acceptable salt thereof, or a pharmaceutically acceptable solvate of any of the foregoing.

A dosage regimen employing oral administration of dosage forms provided by the present disclosure may be developed to maintain a concentration of (R)-baclofen in the blood of a patient, which is greater than a minimum therapeutically effective concentration and less than a minimum adverse concentration for a prolonged period of time. In certain embodiments, a minimum therapeutically effective concentration of (R)-baclofen may range from about 1 ng/mL to about 200 ng/mL, and in certain embodiments, can range from about 10 ng/mL to about 100 ng/mL. In certain embodiments, a minimum adverse concentration can range from about 200 ng/mL to about 2,000 ng/mL, and in certain embodiments, can range from about 500 ng/mL to about 1,000 ng/mL. A minimum therapeutic concentration and a minimum adverse concentration will depend on a number of factors such as the disease being treated, the severity of the disease, the intended clinical outcome, the condition of the patient being treated, and so forth. Such regimens may employ repeated dosing of one or more dosage forms provided by the present disclosure. An appropriate interval of dosing may depend, for example, on the amount of (R)-baclofen such as compound (4) in the dosage form, the composition of the dosage form, the release characteristics of compound (4) from the dosage form, the disease being treated, the condition of the patient, the potential adverse effects, and the judgment of the prescribing physician. Dosage regimens may include repeated administration of the same dosage form at each interval or different dosage forms at different intervals. For example, a twice-daily dosage regimen can include the administration of a first dosage form in the morning, and a second dosage form in the evening.

Dosage forms provided by the present disclosure further include dosage forms that are bioequivalent to the dosage forms disclosed herein, in terms of both rate and extent of absorption, for example as defined by the U.S. Food and Drug Administration and discussed in “Guidance for Industry—Bioavailability and Bioequivalence Studies for Orally Administered Drug Products” (2003).

### Dissolution Profiles of Tablet Dosage Forms

Dosage forms provided by the present disclosure comprising compound (4) may be characterized, in part, by their in vitro dissolution profile. Methods for determining dissolution profiles of dosage forms are well known to those skilled in the pharmaceutical arts. Standard methodologies set forth in the U.S. Pharmacopeia may be used. For example, a dissolution profile may be measured in either a U.S. Pharmacopeia Type I Apparatus (baskets) or a U.S. Pharmacopeia Type II Apparatus (paddles).

Using the latter method, dissolution, or release, profiles of (R)-baclofen prodrug dosage forms provided by the present disclosure may be determined by immersing dosage forms in a 10 mM monobasic potassium phosphate buffer (KH₂PO₄) at pH 7.4, and a temperature of 37° C. The dissolution medium is agitated at 75 rpm (USP, Type II). Samples are withdrawn from the dissolution medium at intervals and the content of compound (4) and (R)-baclofen in the dissolution medium determined using reversed phase HPLC.

Dosage forms provided by the present disclosure comprising a (R)-baclofen prodrug can be formulated so that a desired dissolution profile is achieved by including a single population of particles or at least two particle populations in a single dosage form. Alternatively, in certain embodiments, a desired dissolution profile can be achieved by employing more than one dosage form in which the separate dosage forms comprise particles with different release characteristics.

In certain embodiments, release of a (R)-baclofen prodrug from the population (a) particles can exhibit the following in vitro dissolution profile in 10 mM monobasic potassium phosphate buffer at pH 7.4 and 37° C. agitated at 75 rpm (USP, Type II): about 20% of the (R)-baclofen prodrug is released within about 1.5 hours; about 50% of the (R)-baclofen prodrug is released within about 2.5 hours; and about 80% of the (R)-baclofen prodrug is released within about 4 hours.

In certain embodiments, release of (R)-baclofen prodrug from the population (b) particles can exhibit the following in vitro dissolution profile in 10 mM monobasic potassium phosphate buffer at pH 7.4 and 37° C. agitated at 75 rpm (USP, Type II): about 20% of the (R)-baclofen prodrug is released within about 13 minutes; about 50% of the (R)-baclofen prodrug is released within about 20 minutes; and about 80% of the (R)-baclofen prodrug is released within about 25 minutes; and substantially no (R)-baclofen prodrug is released from the population (b) particles in 10 mM potassium monophosphate buffer at pH 6.0 and 37° C. after at least about 60 minutes.

In certain embodiments, release of (R)-baclofen prodrug from population (c) particles can exhibit the following in vitro dissolution profile in 10 mM monobasic potassium phosphate buffer (KH₂PO₄) at pH 7.4 and 37° C. agitated at 75 rpm (USP, Type II): about 20% of the (R)-baclofen prodrug is released within about 2.5 minutes; about 50% of the (R)-baclofen prodrug is released within about 6 minutes; and about 80% of the (R)-baclofen prodrug is released within about 10 minutes.

In certain embodiments, release of compound (4) from controlled release particles exhibits an in vitro dissolution profile in 10 mM monobasic potassium phosphate buffer at pH 7.4 and 37° C. agitated at 75 rpm (USP, Type II) in which from about 35% to about 45% of compound (4) is released within about 4 hours; from about 60% to about 80% of compound (4) is released within about 7.6 hours; and from about 85% to about 95% of compound (4) is released within about 13 hours. In certain embodiments, release of compound (4) from controlled release particles exhibits an in vitro dissolution profile in 10 mM monobasic potassium phosphate buffer at pH 7.4 and 37° C. agitated at 75 rpm (USP, Type II) in which from about 20% of compound (4) is released within about 4 hours; from about 70% of compound (4) is released within about 7.6 hours; and from about 90% of compound (4) is released within about 13 hours.

Certain embodiments provided by the present disclosure provide oral pharmaceutical dosage forms of an (R)-baclofen prodrug, comprising a combination of at least two particle populations, wherein at least one of the two particle populations is chosen from (a) and (b): (a) a population of (R)-baclofen prodrug-containing particles, the particles when placed in an aqueous solution releasing the (R)-baclofen prodrug into the solution with a release profile that is independent of the solution pH; and (b) a population of (R)-baclofen prodrug-containing particles, the particles when placed in an aqueous solution releasing the (R)-baclofen prodrug into the solution with a release profile that is dependent on the solution pH, wherein the dosage form provides a therapeutically effective concentration of (R)-baclofen in blood and/or plasma of a patient for a continuous period of time after the dosage form is orally administered to the patient. In certain of such embodiments, the R)-baclofen prodrug is (3R)-4-{[(1S)-2-methyl-1-(2-methylpropanoyloxy)propoxy]carbonylamino}-3-(4-chlorophenyl)butanoic acid or a pharmaceutically acceptable salt or solvate thereof, which in certain embodiments can be in a crystalline form.

In certain embodiments of oral dosage forms of an (R)-baclofen prodrug comprising a combination of at least two particle populations, the dosage forms comprise particle populations (a) and (b).

In certain embodiments of oral dosage forms of an (R)-baclofen prodrug comprising a combination of at least two particle populations, the dosage forms can comprise more than one particle population (a) and/or more than one particle population (b) in which each of the more than one particle populations (a) has a different release profile than the other particle populations (a), and each of the more than one particle populations (b) has a different release profile than the other particle populations (b). For example, a dosage form can comprise a first particle population (a) having a first release profile that is independent of the solution pH, and a second particle population (a) having a second release profile that is independent of the solution pH, wherein the second release profile is different than the first release profile. As another example, a dosage form can comprise a first particle population (b) having a third release profile that is dependent on the solution pH, and a second particle population (b) having a fourth release profile that is dependent on the solution pH, wherein the fourth release profile is different than the third release profile.

In certain embodiments of oral dosage forms of an (R)-baclofen prodrug comprising a combination of at least two particle populations, the dosage forms further comprise a population of (R)-baclofen prodrug-containing particles (population (c) particles), the particles when placed in an aqueous solution releasing substantially all of the (R)-baclofen prodrug into the solution within about 1 hour of being placed in the solution.

Oral dosage forms provided by the present disclosure can comprise a combination of particle populations such as particle populations (a) and (c), particle populations (a) and (b), or particle populations (a), (b), and (c).

### Therapeutic Uses

Sustained release oral dosage forms provided by the present disclosure can be administered to a patient suffering from any disease or disorder for which the parent drug, (R)-baclofen, is known, believed to be, or hereafter discovered to be therapeutically effective. Indications for which (R)-baclofen has been prescribed, and hence for which the dosage forms provided by the present disclosure are also effective, include spasticity, gastro-esophageal reflux disease, narcotic addiction or abuse, alcohol addiction or abuse, nicotine addiction or abuse, emesis, cough, neuropathic pain, and/or musculoskeletal pain. The dosage forms can also be administered to a patient as a preventive measure against the diseases or disorders disclosed herein. Thus, the dosage forms can be administered as a preventive measure to a patient having a predisposition for spasticity, gastro-esophageal reflux disease, narcotic addiction or abuse, alcohol addiction or abuse, nicotine addiction or abuse, emesis, cough, neuropathic pain, and/or musculoskeletal pain.

The suitability of an oral dosage form provided by the present disclosure in treating the above-listed diseases and conditions can be determined by methods described in the art.

The efficacy of dosage forms comprising (R)-baclofen prodrugs provided by the present disclosure for treating spasticity can be assessed in animal models of neuropathic pain and in clinical trials. Animal models of spasticity are known (See e.g., Eaton, J Rehab Res Dev 2003, 40(4), 41-54; Kakinohana et al., Neuroscience 2006, 141, 1569-1583; Ligresti et al., British J Pharm 2006, 147, 83-91; Zhang et al., Chinese J Clin Rehab, 2006, 10(38), 150-151; Hefferan et al., Neuroscience Letters 2006, 403, 195-200; and Li et al., J Neurophysiol 2004, 92, 2694-2703. Randomized double-blind placebo-controlled clinical trials for evaluating spasticity treatments are described, for example, in Priebe et al., Spinal Cord 1997, 35(3), 171-5; Gruenthal et al., Spinal Cord 1997, 35(10), 686-9; and Tuszynski et al., Spinal Cord 2007, 45, 222-231; and Steeves et al., Spinal Cord 2007, 45, 206-221 for examples of the conduct and assessment of clinical trials for spasticity caused by spinal cord injury.

The efficacy of dosage forms comprising (R)-baclofen prodrugs provided by the present disclosure for treating gastroesophageal reflux disease can be assessed in animal models of neuropathic pain and in clinical trials such as disclosed, for example, in Blackshaw et al., Am J Physiol 1999, 277, G867-G874; and Lehamnn et al., Eur J Pharmacology 2000, 403, 163-167.

The efficacy of dosage forms comprising (R)-baclofen prodrugs provided by the present disclosure for treating emesis can be assessed in animal models of neuropathic pain and in clinical trials such as disclosed, for example, in Oliver et al., Chem-Biol Interaction 1989, 69, 353-357.

The efficacy of dosage forms comprising (R)-baclofen prodrugs provided by the present disclosure for treating cough can be assessed in animal models of neuropathic pain and in clinical trials such as disclosed, for example, in Bolser et al., Br J Pharmacol 1993, 110, 491-495; Lewis et al., Pulmonary Pharmacology & Therapeutics 2007, 20, 325-333; and Chan et al., Eur J Pharmacology 2007, 559, 196-201.

The efficacy of dosage forms comprising (R)-baclofen prodrugs provided by the present disclosure for treating narcotic addiction and abuse can be assessed in animal models of neuropathic pain and in clinical trials such as disclosed, for example, in Heinzerling et al., Drug Alcohol Dependence 2006, 85, 177-184; Haney et al., Neuropsychopharmacology 2006, 31, 1814-1821; and Spano et al., Neuropharmacology 2007, 52, 1555-1562.

The efficacy of dosage forms comprising (R)-baclofen prodrugs provided by the present disclosure for treating alcohol addiction and abuse can be assessed in animal models of neuropathic pain and in clinical trials such as disclosed, for example, in Flannery et al., Alcoholism: Clin Experimental Res 2004, 28(10), 1517-1523.

The efficacy of dosage forms comprising (R)-baclofen prodrugs provided by the present disclosure for treating nicotine addiction and abuse can be assessed in animal models of neuropathic pain and in clinical trials such as disclosed, for example, in Paterson et al., Neuropsychopharmacology 2005, 30, 119-128.

The efficacy of dosage forms comprising (R)-baclofen prodrugs provided by the present disclosure for treating one or more types of neuropathic pain can be assessed in animal models of neuropathic pain and in clinical trials (see e.g., Beggs and Salter, Drug Dev Res 2006, 67, 829-301). Useful animal models of neuropathic pain include peripheral nerve injury by ligation or transection include dorsal rhizotomy (Lombard et al., Pain 1979, 6(2), 163-174); spinal nerve ligation (Kim and Chung, Pain 1992, 50, 355-363; and Hwang and Yaksh, Pain 1997, 70, 15-22); sciatic nerve transaction (Wall et al., Pain 1979, 7, 103-111); sciatic nerve cuff (Mosconi and Kruger, Pain 1996, 64, 37-57); partial nerve ligation (Seltzer et al., Pain 1990, 43, 205-218); chronic constriction (Bennett and Xie, Pain 1988, 33, 87-107); rat spinal cord ischemia model (Hao et al., Pain 1991, 45, 175-185; and von Heijne et al., Eur J Pain 2001, 5, 1-10); and spared nerve injury (Decosterd and Woolf, Pain 2000, 87, 149-158).

The efficacy of prodrugs of GABA_{B} agonists provided by the present disclosure for treating one or more types of musculoskeletal pain can be assessed in animal models of neuropathic pain and in clinical trials. For example, Kehl et al., disclose an animal model of muscle hyperplasia that employs intramuscular injection of carrageenan as useful for assessing the mechanisms and management of musculoskeletal pain (Kehl et al., Pain 2000, 85, 333-343).

### Dosing

The amount of a (R)-baclofen prodrug that will be effective in the treatment of a particular disease, disorder, or condition disclosed herein will depend, at least in part, on the nature of the disorder or condition, and can be determined by standard clinical techniques known in the art as previously described. In addition, in vitro or in vivo assays can optionally be employed to help identify optimal dosage ranges. The amount of a (R)-baclofen prodrug administered can depend on, among other factors, the subject being treated, the weight of the subject, the severity of the disease or disorder, the manner of administration, and the judgment of the prescribing physician. Dosage ranges and dosing intervals can be determined by methods known to those skilled in the art. Suitable dosage ranges for oral administration of (R)-baclofen are generally about 0.1 mg/day to about 100 mg/day and the dose of a (R)-baclofen prodrug can be adjusted to provide an equivalent molar quantity or mass equivalent dose of (R)-baclofen. In certain embodiments, a dose can comprise a mass equivalent of (R)-baclofen ranging from about 0.1 mg to about 100 mg, in certain embodiments, from about 0.5 mg to about 80 mg, and in certain embodiments, from about 2 mg to about 40 mg. The dose of a (R)-baclofen prodrug and appropriate dosing intervals can be selected to maintain a sustained therapeutically effective concentration of (R)-baclofen in the blood and/or plasma of a patient, and in certain embodiments, without exceeding a minimum adverse concentration.

In certain embodiments, dosage forms provided by the present disclosure can be administered once per day such as a once-daily dosage from comprising a mass equivalent of (R)-baclofen ranging from about 0.5 mg to about 50 mg, in certain embodiments, twice per day such as a twice-daily dosage from comprising a mass equivalent of (R)-baclofen ranging from about 0.5 mg to about 50 mg, and in certain embodiments at intervals greater than once per day. Dosing can be provided alone or in combination with other drugs and can continue as long as required for effective treatment of the disease or disorder. Dosing includes administering a dosage from to a mammal, such as a human, in a fed or fasted state.

A dose of a (R)-baclofen prodrug such as compound (4) can be adjusted to provide an equivalent molar quantity or mass equivalent dose of (R)-baclofen. Therapeutically effective doses of (R)-baclofen are generally from about 0.15 mg to about 2.5 mg per kilogram body weight per day. In certain embodiments, a dose can comprise a mass equivalent of (R)-baclofen ranging from about 0.1 mg to about 100 mg, in certain embodiments, from about 0.5 mg to about 80 mg, and in certain embodiments, from about 2 mg to about 40 mg. The dose of compound (4) and appropriate dosing intervals can be selected to maintain a sustained therapeutically effective concentration of (R)-baclofen in the blood of a patient, and in certain embodiments, without exceeding a minimum adverse concentration.

In certain embodiments, dosage forms provided by the present disclosure may be administered once per day, twice per day, and in certain embodiments at intervals greater than once per day. Dosing may be provided alone or in combination with other drugs and may continue as long as required for effective treatment of the disease. Dosing includes administering a dosage form to a mammal, such as a human, in a fed or fasted state.

### Combination Therapy

Dosage forms provided by the present disclosure may further comprise one or more pharmaceutically active compounds in addition to a (R)-baclofen prodrug. Such compounds may be provided to treat the same disease or a different disease than the disease being treated with the (R)-baclofen prodrug.

In certain embodiments, a (R)-baclofen prodrug may be used in combination with at least one other therapeutic agent. In certain embodiments, compound (4) may be administered to a patient together with another compound for treating movement disorders such as spasticity, digestive disorders such as gastro-esophageal reflux disease and emesis, or addictive or abuse disorders such as nicotine addiction or abuse, alcohol addiction or abuse, narcotic addiction or abuse, cough, neuropathic pain, or musculoskeletal pain. In certain embodiments, the at least one other therapeutic agent may be a different (R)-baclofen prodrug. A (R)-baclofen prodrug and the at least one other therapeutic agent may act additively or, and in certain embodiments, synergistically. The at least one additional therapeutic agent may be included in the same dosage form comprising a (R)-baclofen prodrug or may be in a separate dosage form. Accordingly, methods provided by the present disclosure can further include, in addition to administering a (R)-baclofen prodrug, administering one or more therapeutic agents effective for treating the same or different disease than the disease being treated by a (R)-baclofen prodrug. Methods provided by the present disclosure include administration of a (R)-baclofen prodrug and one or more other therapeutic agents provided that the combined administration does not inhibit the therapeutic efficacy of a (R)-baclofen prodrug and/or does not produce adverse combination effects.

In certain embodiments, dosage forms comprising a (R)-baclofen prodrug may be administered concurrently with the administration of another therapeutic agent, which may be part of the same dosage form as, or in a different dosage form than that comprising a (R)-baclofen prodrug. A (R)-baclofen prodrug may be administered prior or subsequent to administration of another therapeutic agent. In certain embodiments of combination therapy, the combination therapy may comprise alternating between administering a (R)-baclofen prodrug and a composition comprising another therapeutic agent, e.g., to minimize adverse drug effects associated with a particular drug. When a (R)-baclofen prodrug is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising a (R)-baclofen prodrug may be administered with one or more substances to enhance, modulate and/or control release, bioavailability, therapeutic efficacy, therapeutic potency, stability, and the like of a (R)-baclofen prodrug. For example, to enhance the therapeutic efficacy of a (R)-baclofen prodrug or its metabolite, (R)-baclofen, a (R)-baclofen pro drug may be co-administered with a dosage form comprising a (R)-baclofen prodrug or may comprise one or more active agents to increase the absorption or diffusion of a (R)-baclofen prodrug or (R)-baclofen from the gastrointestinal tract to the systemic circulation, and/or to inhibit degradation of a (R)-baclofen prodrug or (R)-baclofen in the blood of a patient. In certain embodiments, a (R)-baclofen prodrug may be co-administered with a dosage form comprising a (R)-baclofen prodrug or an active agent having pharmacological effects that enhance the therapeutic efficacy of a (R)-baclofen prodrug.

Additionally, oral dosage forms comprising a (R)-baclofen prodrug provided by the present disclosure may be used in combination with other drugs that are themselves known to cause spasticity, gastro-esophageal reflux disease, narcotic addiction or abuse, alcohol addiction or abuse, nicotine addiction or abuse, emesis, cough, neuropathic pain, and/or musculoskeletal pain as an adverse effect, thereby preventing or reducing the occurrence of such adverse effects.

Particles and/or dosage forms provided by the present disclosure can further comprise one or more pharmaceutically active compounds other than a (R)-baclofen prodrug. Such compound may be provided to treat the same condition or a different condition being treated with a (R)-baclofen prodrug.

Methods provided by the present disclosure can further include, in addition to administering a (R)-baclofen prodrug, administering one or more therapeutic agents effective for treating the same or different disease, disorder, or condition as the disease disorder, or condition being treated by a (R)-baclofen prodrug. Methods provided by the present disclosure include administration of a (R)-baclofen prodrug and one or more other therapeutic agents provided that the combined administration does not inhibit the therapeutic efficacy of a (R)-baclofen prodrug and/or does not produce adverse combination effects.

In certain embodiments, a (R)-baclofen prodrug can be administered concurrently with the administration of another therapeutic agent, which can be part of the same dosage form as, or in a different dosage form than the dosage form containing a (R)-baclofen prodrug. In certain embodiments, a (R)-baclofen prodrug can be administered prior or subsequent to administration of another therapeutic agent. In certain embodiments of combination therapy, the combination therapy comprises alternating between administering a (R)-baclofen prodrug and a composition comprising another therapeutic agent, e.g., to minimize adverse drug effects associated with a particular drug. When a (R)-baclofen prodrug is administered concurrently with another therapeutic agent that potentially can produce an adverse drug effect including, but not limited to, toxicity, the other therapeutic agent can advantageously be administered at a dose that falls below the threshold at which the adverse drug reaction is elicited.

In certain embodiments, a (R)-baclofen prodrug can be administered with one or more substances to enhance, modulate and/or control release, bioavailability, therapeutic efficacy, therapeutic potency, stability, and the like of a (R)-baclofen prodrug. For example, to enhance the therapeutic efficacy of a (R)-baclofen prodrug or its metabolite, (R)-baclofen, a (R)-baclofen prodrug can be co-administered with one or more active agents to increase the absorption or diffusion of a (R)-baclofen prodrug or (R)-baclofen from the gastrointestinal tract to the systemic circulation, or to inhibit degradation of a (R)-baclofen prodrug in the systemic circulation or blood and/or plasma of a patient. In certain embodiments, a (R)-baclofen prodrug can be co-administered with an active agent having pharmacological effects that enhance the therapeutic efficacy of a (R)-baclofen prodrug.

Methods provided by the present disclosure include administering one or more (R)-baclofen prodrugs and one or more other therapeutic agents provided that the combined administration does not inhibit the therapeutic efficacy of the one or more (R)-baclofen prodrugs and/or other therapeutic agent and/or does not produce adverse combination effects.

In certain embodiments, (R)-baclofen prodrugs provided by the present disclosure and pharmaceutical compositions thereof may be administered to a patient for treating a movement disorder such as spasticity in combination with a therapy or another therapeutic agent known or believed to be effective in treating a movement disorder such as spasticity. Examples of drugs for treating movement disorders such as spasticity include levodopa, mild sedatives such as benzodiazepines including alprazolam, chlordiazepoxide, clonazepam, clorazepate, diazepam, lorazepam, and oxazepam; muscle relaxants such as baclofen, anticholinergic drugs such as trihexyphenidyl and diphenhydramine; antipsychotics such as chlorpromazine, fluphenazine, haloperidol, loxapine, mesoridazine, molindone, perphenazine, pimozide, thioridazine, thiothixene, trifluoperazine, aripiprazole, clozapine, olanzapine, quetiapine, risperidone, and ziprasidone; and antidepressants such as amitriptyline.

In certain embodiments, oral dosage forms comprising (R)-baclofen prodrugs provided by the present disclosure and pharmaceutical compositions thereof may be administered to a patient for treating a gastrointestinal disorder such as gastroesophageal reflux disease in combination with a therapy or another therapeutic agent known or believed to be effective in treating a gastrointestinal disorder such as gastroesophageal reflux disease. Examples of drugs for treating gastrointestinal disorders such as gastro-esophageal reflux disease include H2 inhibitors such as cimetidine, famotidine, nizatidine, and ranitidine; proton pump inhibitors such as omeprazole, lansoprazole, pantoprazole, rabeprazole, and exomeprazole; and prokinetics such as cisparide, bethanechol, and metoclopramide.

In certain embodiments, oral dosage forms comprising (R)-baclofen prodrugs provided by the present disclosure and pharmaceutical compositions thereof may be administered to a patient for treating emesis in combination with a therapy or another therapeutic agent known or believed to be effective in treating emesis. Examples of drugs for treating emesis (nausea and vomiting) include benzamines such as metoclopramide; phenothiazines such as prochlorperazine, perphenazine, chlorpromazine, promethazine, and thiethylperazine; butyrophenones such as droperidol and haloperidol; dopamine 2 antagonists such as metoclorpamide; 5-HT3 antagonists such as ondansetron, granisetron, dolasetron, palonosetron; NK-1 receptor antagonists such as aprepitant, corticosteroids such as dexamethazone; antihistamines such as diphenhydramine and hydroxyzine; cannabinoids such as dronabinol; and benzodiazepines such as lorazepam, midazolam, alprazolam, and olanzapine

In certain embodiments, oral dosage forms comprising (R)-baclofen prodrugs provided by the present disclosure and pharmaceutical compositions thereof may be administered to a patient for treating alcohol addiction or abuse in combination with a therapy or another therapeutic agent known or believed to be effective in treating alcohol addiction or abuse. Examples of drugs for treating alcohol addiction or abuse include disulfuram, naltrexone, clonidine, methadone, 1-alpha-acetylmethadol, buprenorphine, and bupropion.

In certain embodiments, oral dosage forms comprising (R)-baclofen prodrugs provided by the present disclosure and pharmaceutical compositions thereof may be administered to a patient for treating narcotic addiction or abuse in combination with a therapy or another therapeutic agent known or believed to be effective in treating narcotic addiction or abuse. Examples of drugs for treating narcotic addiction or abuse include buprenorphine, tramadol, methadone, and naltrexone.

In certain embodiments, oral dosage forms comprising (R)-baclofen prodrugs provided by the present disclosure and pharmaceutical compositions thereof may be administered to a patient for treating nicotine addiction or abuse in combination with a therapy or another therapeutic agent known or believed to be effective in treating nicotine addiction or abuse. Examples of drugs for treating nicotine addiction or abuse include bupropion, clonidine, and nicotine.

In certain embodiments, oral dosage forms comprising (R)-baclofen prodrugs provided by the present disclosure and pharmaceutical compositions thereof may be administered to a patient for treating cough in combination with a therapy or another therapeutic agent known or believed to be effective in treating cough. Examples of drugs for treating cough include dixtromethorphan, guaifenesin, hydrocodone, benzonatate, diphenhydramine, pseudoephedrine, acetaminophen, and carbinoxamine.

In certain embodiments, oral dosage forms comprising (R)-baclofen prodrugs provided by the present disclosure and pharmaceutical compositions thereof may be administered to a patient for treating neuropathic pain in combination with a therapy or another therapeutic agent known or believed to be effective in treating neuropathic pain. Examples of drugs useful for treating neuropathic pain include opioid analgesics such as morphine, codeine, fentanyl, meperidine, methadone, propoxyphene, levorphanol, hydromorphone, oxycodone, oxymorphone, tramadol and pentazocine; nonopioid analgesics such as aspirin, ibuprofen, ketoprofen, naproxen, and acetaminophen; non-steroidal anti-inflammatory drugs such as aspirin, choline magnesium trisalicylate, diflunisal, salsalate, celecoxib, rofecoxib, valdecoxib, diclofenac, etodolac, fenoprofen, flubiprofen, ibuprofen, indomethacin, ketoprofen, ketorolac, meclofanamate, mefenamic acid, meloxicam, nabumetone, naproxen, oxaprozin, piroxicam, sulindac, and tometin; antiepileptics such as gabapentin, pregabalin, carbamazepine, phenyloin, lamotrigine, and topiramate; antidepressants such as duloxetine, amitriptyline, venlafaxine, nortryptyline, imipramine, and desipramine; local anesthetics such as lidocaine, and mexiletine; NMDA receptor antagonists such as dextropethorphan, memantine, and ketamine; N-type calcium-channel blockers such as ziconotide; vanilloid receptor-1 modulators such as capsaicin; cannabinoid receptor modulators such as sativex; neurokinin receptor antagonists such as lanepitant; other analgesics such as neurotropin; and other drugs such as desipramine, clonazepam, divalproex, oxcarbazepine, divalproex, butorphanol, valdecoxib, vicoprofen, pentazocine, propoxyhene, fenoprofen, piroxicam, indometnacin, hydroxyzine, buprenorphine, benzocaine, clonidine, flurbiprofen, meperidine, lacosamide, desvenlafaxine, and bicifadine.

In certain embodiments, oral dosage forms comprising (R)-baclofen prodrugs provided by the present disclosure and pharmaceutical compositions thereof may be administered to a patient for treating musculoskeletal pain in combination with a therapy or another therapeutic agent known or believed to be effective in treating musculoskeletal pain. Examples of drugs useful for treating musculoskeletal pain include cyclobenzaprine, dantrolene, methocarbamrol, orphenadrine, tizanidrine, metaxalone, carisoprodol, chlorphenesin, chlorzoxazone, alprazolam, bromazepam, chlordiazepoxide, clorazepate, diazepam, flunitriazepam, lorazepam, medazepam, midazolam, oxazepam, prazepam, triazolam, temazepam, and botulinum toxin.

In certain embodiments, oral dosage forms comprising (R)-baclofen prodrugs provided by the present disclosure and pharmaceutical compositions thereof may be administered to a patient for treating low back pain in combination with a therapy or another therapeutic agent known or believed to be effective in treating low back pain. Examples of drugs useful for treating low back pain include NSAIDs such as aspirin, naproxen, and ibuprofen; anticonvulsants, antidepressants such as amitriptyline and desipramine; and opioids such as codeine, oxycodone, hydrocodone, and morphine. In certain embodiments, any of the drugs useful for treating neuropathic pain may be coadminstered with a prodrug of a GABAB agonist for treating low back pain. Therapies for low back pain include the use of cold and hot compresses, bed rest, exercise, spinal manipulation, acupuncture, biofeedback, interventional therapy, traction, transcutaneous electrical nerve stimulation, ultrasound, vertebroplasty, kyphoplasty, discectomy, foraminotomy, intradiscal electrothermal therapy, nucleoplasty, radiofrequency lesioning, spinal fusion, and spinal laminectomy.

### EXAMPLES

The following examples describe in detail preparation and characterization of oral dosage forms comprising prodrugs of (R)-baclofen and methods of using such dosage forms. It will be apparent to those skilled in the art that many modifications, both to materials and methods, may be practiced without departing from the scope of the disclosure.

### Example 1

### Controlled Release Capsules

Immediate release (IR) particles comprising (R)-baclofen prodrug, (3R)-4-{[(1S)-2-methyl-1-(2-methylpropanoyloxy)propoxy]carbonylamino}-3-(4-chlorophenyl)butanoic acid (4), were prepared by coating cores comprising the (R)-baclofen prodrug (4) with a pH independent release coating. 20/25 mesh sugar spheres (sugar spheres NF, Paulaur, Cranbury, N.J.) were added to a fluid-bed coater bowl and heated to 29-31° C. A coating formulation was prepared by dissolving (R)-baclofen prodrug (4) and binder (Plasdone® K29/32 Povidone, USP/NF, ISP Corporation) in 409 gm of a 50:50 mixture of isopropyl alcohol and acetone. The coating formulation comprising (R)-baclofen prodrug (4) was sprayed onto the sugar spheres while maintaining the outlet temperature at 29-31° C. to form the immediate-release cores. The amounts of the components forming the immediate-release cores are provided in Table 1.

TABLE 1
Composition of Immediate-Release Cores

| | Amount/Capsule | % Composition | Ingredient |
|---|---|---|---|
| Component | (mg) | (w/w) | category |
| Compound (4) | 2.00 | 2.64 | Drug |
| | | | substance |
| 20/25 Sugar sphere, NF | 73.65 | 97.21 | Inert core |
| Plasdone ® K29/32 | 0.11 | 0.15 | Binder |
| Povidone USP/NF | | | |
| Isopropyl alcohol, USP | — | — | Solvent |
| Acetone, NF | — | — | Solvent |
| Total | 75.76 | 100.00 | — |

pH independent release particles were prepared by coating the immediate release cores with a pH independent release polymer. The cores were spray coated with a mixture comprising which 9.7 gm ammonioalkyl methacrylic acid copolymer type A (Eudragit® RL 100, Rohm Pharma) and 0.3 gm glyceryl monostearate were dissolved in 125 gm of a 60:40 mixture of isopropyl alcohol and acetone.

The controlled release particles, coated with the pH independent release polymer, were then loaded into size #0 hydroxypropylmethyl cellulose (HPMC) capsules in a quantity to provide 10 mg of compound (4), equivalent to 5.34 gm of (R)-baclofen. The pH independent release particles were then loaded into HPMC capsules. The relative amounts of the components forming controlled release capsules are provided in Table 2.

TABLE 2
Composition of Capsules Comprising pH Independent Release Particles

| | Amount/ | % | |
|---|---|---|---|
| | Capsule | Composition | Ingredient |
| | (mg) | (w/w) | category |
| IR Cores/Compound (4) | 353.16 | 82.64 | Drug substance |
| | | | coated beads |
| Ammonioalkyl | 71.97 | 16.84 | pH-independent |
| Methacrylate | | | |
| Copolymer Type A | | | release |
| (Eudragit ® RL 100), | | | controlling |
| USP, NF | | | polymer |
| Glyceryl Monostearate, | 2.22 | 0.52 | Antistatic Agent |
| USP, NF | | | |
| Size #0 white, opaque | — | — | Capsule Shell |
| HPMC capsule shell | | | |
| Total | 427.35 | 100.00 | — |

### Example 2

### Composition of pH-Dependent Release Capsules

pH-dependent release capsules were manufactured using essentially the same procedure as in Example 1 using a coating mixture in which 9.7 gm methacrylic acid copolymer type B (Eudragit™ S 100, Rohm Pharma) and 0.3 g glyceryl monostearate were dissolved in 125 mL of a 60:40 mixture of isopropyl alcohol and acetone. The relative amounts of the components forming an pH dependent-release capsule are provided in Table 2.

TABLE 2
Composition of pH-Dependent Release Capsules

| | Amount/ | % | |
|---|---|---|---|
| | Capsule | Composition | Ingredient |
| | (mg) | (w/w) | category |
| IR Beads/Compound (4) | 82.29 | 89.28 | Drug substance |
| | | | coated beads |
| Methacrylic acid copolymer | 9.58 | 10.39 | pH-dependent |
| Type B (Eudragit ™ S 100), | | | release control |
| NF | | | polymer |
| Glyceryl Mono Stearate, | 0.30 | 0.33 | Lubricant |
| USP, NF | | | |
| Isopropyl alcohol, USP | — | — | Solvent |
| Acetone, NF | — | — | Solvent |
| Total Weight | 92.17 | 100.00 | |

### Example 3

### In Vitro Dissolution Profiles

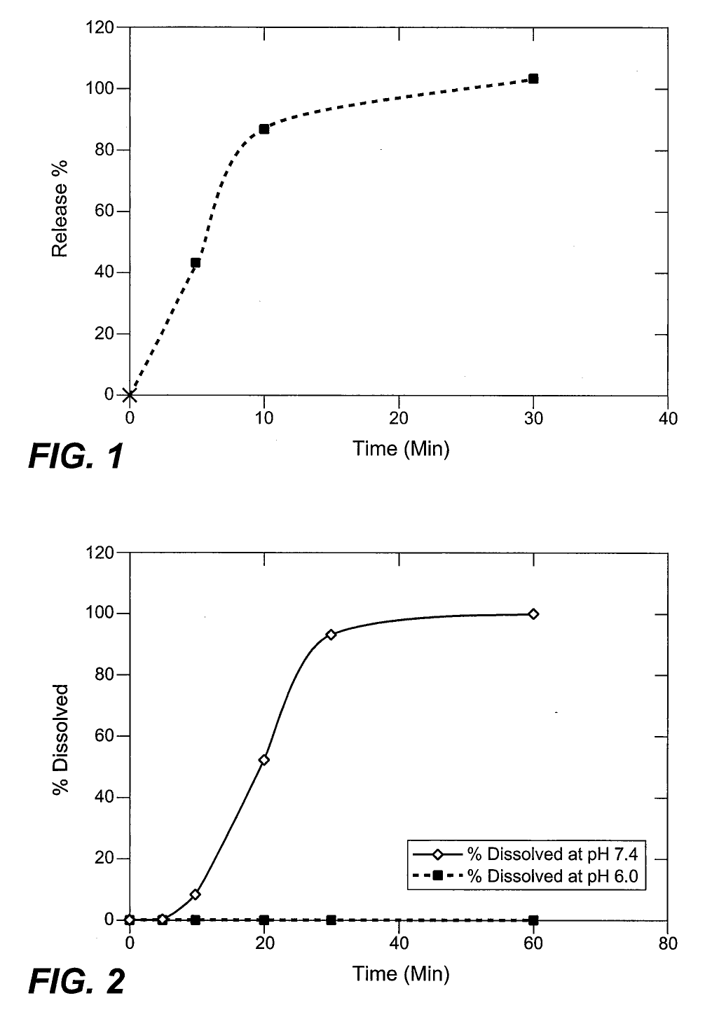

In vitro dissolution profiles for the dosage forms prepared according to Examples 1-2 were determined according to USP Method 2 (Type II, paddle method) using a Model Evolution 4300-7 Vessel USP II bath (Distek Inc., New Brunswick, N.J.). Dosage forms were placed into a dissolution vessel containing 500 mL of 10 mM monobasic potassium phosphate buffer (KH₂PO₄) at pH 7.4, 37° C. The dissolution medium was agitated at 75 rpm (USP, Type II). Samples were withdrawn at intervals up to about 20 hours and the content of compound (4) in solution was determined by reverse phase HPLC using a C18 column and a phosphate buffer/acetonitrile/water isocratic mobile phase with photodiode detection at 210 nm. An in vitro dissolution profile for controlled release capsules prepared according to Example 1 is shown in FIG. 1.

As shown in FIG. 1, immediate-release particles comprising (R)-baclofen prodrug (4) released more than 80% of compound (4) contained within the capsule within 10 minutes.

As shown in FIG. 2, pH-dependent release particles comprising (R)-baclofen prodrug (4) released more than 80% of compound (4) within 25 minutes at pH 7.4, and substantially no prodrug was released after at least about 60 minutes at pH 6.0.

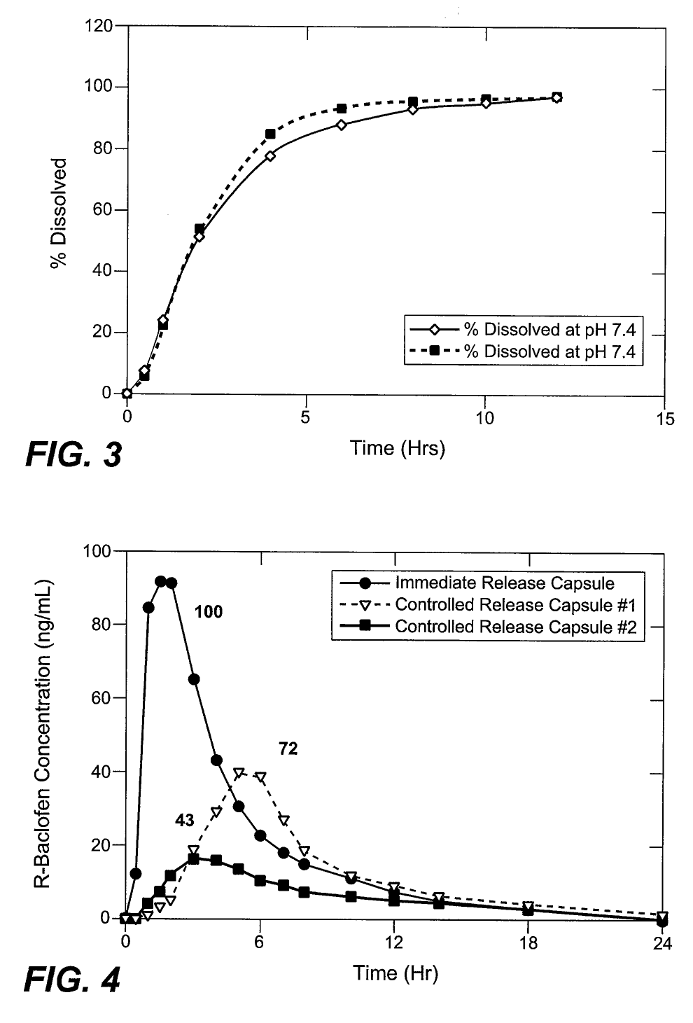

As shown in FIG. 3, pH-independent release particles comprising (R)-baclofen prodrug (4) released more than 80% of compound (4) within about 4 hours.

### Example 4

### Pharmacokinetics of (R)-Baclofen in Dogs

Dosage forms comprising compound (4) were administered by oral gavage to groups of four adult male Beagle dogs (weight approx 8 kg) at a dose of 10 mg compound (4). The dogs were fasted overnight before the study and for 4 hours post-dosing. Blood samples (1 mL) were obtained via the femoral vein at intervals over 24 hours after oral dosing. Blood was quenched immediately using acetonitrile with 1% formic acid and then frozen at −20° C. until analyzed.

The concentration of (R)-baclofen in quenched whole blood was determined using an API 2000 LC/MS/MS instrument equipped with a Shimadzu binary pump and a Leap CTC autosampler. The column was a Phenomenex Hydro-RP 4.6×50 mm column operating at room temperature. The mobile phases were (A) water with 0.1% formic acid and (B) acetonitrile with 0.1% formic acid. The gradient condition was: 5% B for 0.5 min, then to 95% B in 1.8 min, then maintained at 95% B for 1.2 min. The mobile phase was returned to 5% B for 2 min. A TurbolonSpray source was used on the API 2000. The analysis was done in positive ion mode and an MRM transition of m/z 214/151 was used in the analysis of (R)-baclofen. Ten (10) SL of the blood sample was injected. The peaks were integrated using Analyst™ Software (Agilent Technologies) to provide the concentration of (R)-baclofen in the blood sample.

The pharmacokinetic parameters of capsules comprising pH independent-release particles prepared according to Examples 1-4 at a dose of 10 mg compound (4) following oral administration to dogs are provided in Table 4. The bioavailability is determined relative to 1 mg-eq/kg (R)-baclofen administered intravenously.

TABLE 4
Pharmacokinetics (mean (SD)) of (R)-Baclofen Following Oral
Administration of Dosage Forms Comprising pH-Independent
Release Particles to Dogs at a Dose of 10 mg of Compound (4).

| | Cₘₐₓ | Cₘₐₓ/ | | | AUC_{inf} | F |
|---|---|---|---|---|---|---|
| | (ng/mL) | C₁₂ | Tₘₐₓ (h) | T_{1/2} (h) | (ng · h/mL) | (%) |
| Mean | 41 (9) | 2.2 (—) | 5.0 (2.6) | 4.5 (0.5) | 408 (20) | 42 (8) |

### Example 5

### Oral Bioavailability of (R)-Baclofen in Human Subjects

A randomized, crossover, fed/fasted single-dose study of the safety, tolerability, and pharmacokinetics of oral administration of compound (4) in healthy adult subjects was performed. The oral dosage forms of Examples 1 and 2 were used in this study. Thirty healthy adult volunteers participated in the study. The subjects were separated into 3 cohorts of 10 individuals. Within each cohort, subjects received either one of three dosage forms (8 subjects) or a placebo (2 subjects) in both fasted and fed states in random order. The dosage forms used in this study were capsules comprising immediate release particles prepared substantially according to Example 1 comprising 8 mg of compound (4), capsules comprising pH-dependent release prepared substantially according to Example 2 comprising 8 mg of compound (4), and capsules comprising pH-independent release particles prepared substantially according to Examples 1 and 2 comprising 8 mg of compound (4).

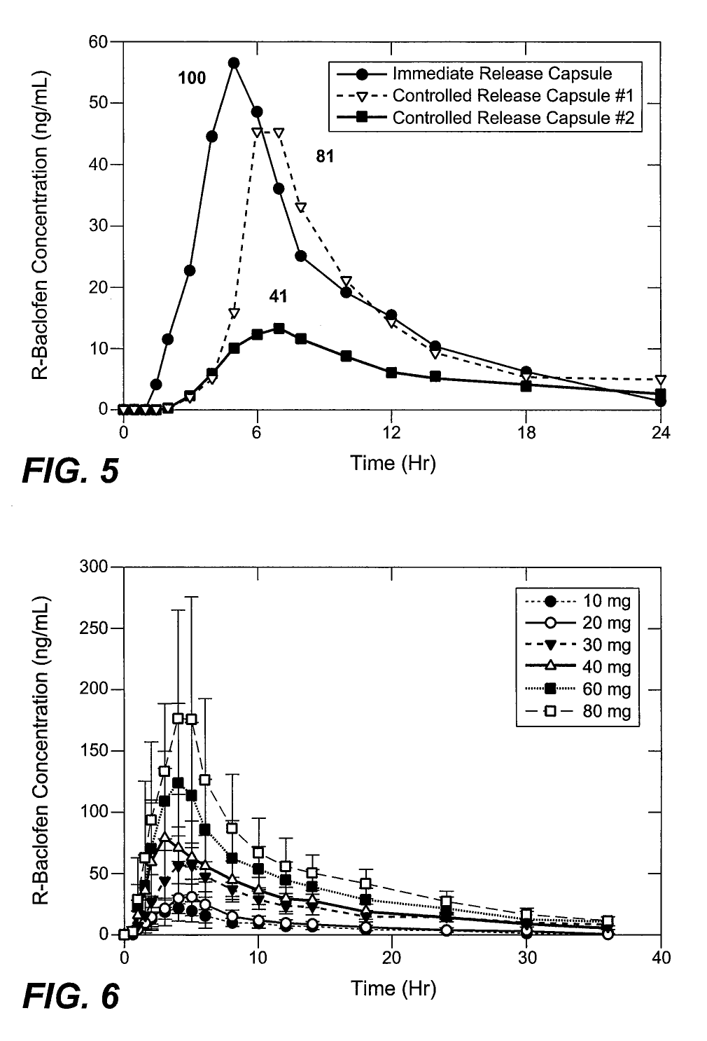

Plasma samples were collected from all subjects prior to dosing, and at 0.5, 1, 1.5, 2, 3, 4, 6, 8, 12, 18, 24, and 36 hours after dosing. Urine samples were collected from all subjects prior to dosing, and complete urine output was obtained at 0-4, 4-8, 8-12, 12-18, 18-24, and 24-36 hours after dosing. Plasma samples were quenched immediately with methanol and stored frozen. Sample aliquots were prepared for analysis of (R)-baclofen and compound (4) using sensitive and specific LC/MS/MS methods. The plasma concentration of (R)-baclofen following administration of a capsule containing immediate release, pH-dependent release, or pH-independent release particles to patients in a fasted or fed state is summarized in Table 5, FIG. 4, and FIG. 5.

TABLE 5
Oral Bioavailability of (R)-Baclofen Following Administration of Oral
Dosage Forms

| | | | | | AUC_{inf} | |
|---|---|---|---|---|---|---|
| Dosage Form | | Cₘₐₓ | Tₘₐₓ | T_{1/2} | (ng · | Fᵣₑₗ* |
| (capsule) | State | (ng/mL) | (h) | (h) | h/mL) | (%) |
| Immediate Release | Fasted | 124 | 1.7 | 4.5 | 443 | 100 |
| | Fed | 60 | 6.0 | 4.4 | 436 | 99 |
| pH-Dependent | Fasted | 55 | 5.1 | 5.9 | 317 | 72 |
| Release | Fed | 49 | 7.5 | 5.2 | 354 | 80 |
| pH-Independent | Fasted | 18 | 3.6 | 10.3 | 189 | 43 |
| Release | Fed | 15 | 6.7 | 10.4 | 180 | 41 |

*AUC relative to the AUC of the immediate release form in fasted subjects.

### Example 6

### Pharmacokinetics of (R)-Baclofen in Human Patients Following Administration of Capsules Comprising pH-Independent Release Particles

The pharmacokinetics of (R)-baclofen in healthy human patients following oral administration of capsules containing pH-independent release particles comprising compound (4) was determined.

Fasted human patients were randomized to receive single oral doses of controlled release (CR) capsules or matching placebo in a double-blind fashion. The study investigated 6 dose levels of compound (4), 10, 20, 30, 40, 60, and 80 mg, in capsules comprising controlled release particles and comprising 10 mg compound (4). Six (6) groups of 10 subjects each were enrolled sequentially (10 subjects per dose level). Eight subjects in each dose group received CR capsules and two received placebo.

Blood samples were collected from patients prior to dosing and at 0.5, 1, 1.5, 2, 3, 4, 5, 6, 8, 10, 12, 14, 18, 24, 30, and 36 hours post-dosing for all treatments. Blood sample aliquots were quenched immediately with methanol to prevent further hydrolysis of compound (4). Blood sample aliquots were stored in a freezer at −70° C. The blood sample aliquots were analyzed for (R)-baclofen and compound (4) in whole blood supernatant using sensitive and specific LC-MS/MS methods.

Concentration data for (R)-baclofen and compound (4) in blood were analyzed by noncompartmental methods using WinNonlin™ Software version 4.1 (Pharsight Corporation, Mountain View, Calif.). Concentration data and pharmacokinetics parameters were plotted using SigmaPlot™ version 9.0 (Systat Software Inc., Point Richmond, Calif.). Actual time points were used for the calculation of pharmacokinetic parameters. The maximum concentration (Cₘₐₓ) and time to Cₘₐₓ(Tₘₐₓ) were obtained by observation. The apparent elimination half-life (T_{1/2}) was determined by linear regression of three or more log-transformed data points in the terminal phase. The area under the concentration versus time curve (AUC) was determined by the linear trapezoidal method using concentration data over the dosing interval. The AUC value extrapolated to infinity (AUC_{inf}) was calculated as:

AUC_{inf}=AUC₀₋ₜₗₐₛₜ+Cₗₐₛₜ/λ_{z}

where tlast is the time of the last quantifiable concentration (Cₗₐₛₜ) and λ_{z} is the rate constant of the apparent terminal elimination phase. Using the data from doses 10, 20, 30, 40, 60, and 80 mg, linear regression models were fit for AUC_{inf} versus dose and for Cₘₐₓ versus dose using SAS™ version 9.1 for Windows (SAS Institute, Cary, N.C.). In both models, the dose effect was parameterized using orthogonal polynomial coefficients for unequally spaced values.

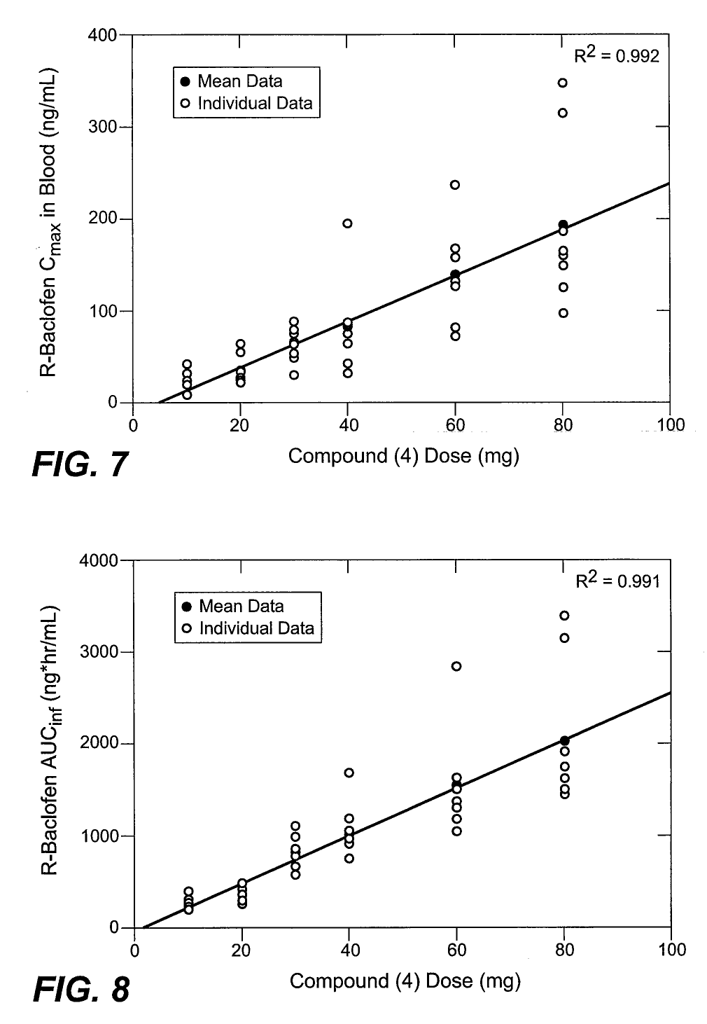

The blood concentration and pharmacokinetic parameters for (R)-baclofen and (R)-baclofen prodrug (4) following oral administration of CR capsules to healthy human patients is shown in FIGS. 6-9, and a summary of the pharmacokinetic parameters for different doses of (R)-baclofen prodrug (4) is provided in Table 6.

TABLE 6
Pharmacokinetic Parameters of (R)-Baclofen Following Oral Administration of
CR Capsules

| | Dose (mg) | | | | | |
|---|---|---|---|---|---|---|
| | 10 | 20 | 30 | 40 | 60 | 80 |
| Cₘₐₓ (ng/mL) | 23 (10) | 35 (17) | 63 (19) | 82 (49) | 139 (56) | 193 (89) |
| Cₘₐₓ/dose | 2.3 | 1.7 | 2.1 | 2.1 | 2.3 | 2.4 |
| Cₘₐₓ/C₁₂ | 2.6 | 2.7 | 2.3 | 2.2 | 2.3 | 2.8 |
| Tₘₐₓ (h) | 5.0 (3.8) | 4.1 (1.1) | 4.8 (0.9) | 4.5 (1.2) | 3.9 (1.1) | 4.0 (1.1) |
| T_{1/2} (h) | 10.3 (3.6) | 9.6 (1.7) | 9.3 (2.7) | 11.3 (4.7) | 10.5 (2.6) | 9.7 (1.0) |
| AUC_{inf} | 243 (66) | 338 (83) | 810 (169) | 1020 (300) | 1540 (603) | 2020 (787) |
| (ng · h/mL) | | | | | | |
| AUC_{inf}/dose | 24 | 17 | 27 | 26 | 26 | 26 |
| F (%) | 31 (7) | 33 (10) | 33 (7) | 28 (9) | 35 (6) | 34 (18) |

Finally, it should be noted that there are alternative ways of implementing the disclosures contained herein. Accordingly, the present embodiments are to be considered as illustrative and not restrictive, and the claims are not to be limited to the details given herein, but may be modified within the scope and equivalents thereof.

What is claimed is:

1. An oral pharmaceutical dosage form of an (R)-baclofen prodrug, comprising:
a combination of at least two particle populations, wherein at least one of the two particle populations is chosen from (a) and (b):
(a) a population of (R)-baclofen prodrug-containing particles, the particles when placed in an aqueous solution releasing the (R)-baclofen prodrug into the solution with a release profile that is independent of the solution pH; and
(b) a population of (R)-baclofen prodrug-containing particles, the particles when placed in an aqueous solution releasing the (R)-baclofen prodrug into the solution with a release profile that is dependent on the solution pH;
wherein the oral dosage form provides a therapeutically effective concentration of (R)-baclofen in plasma of a patient for a continuous period of time after the oral dosage form is orally administered to the patient.

2. The oral dosage form of claim 1, wherein the combination further comprises a particle population (c):
(c) a population of (R)-baclofen prodrug-containing particles, the particles when placed in an aqueous solution releasing substantially all of the (R)-baclofen prodrug into the solution within about 1 hour of being placed in the solution.

3. The oral dosage form of claim 1, wherein the (R)-baclofen prodrug is (3R)-4-{[(1S)-2-methyl-1-(2-methylpropanoyloxy)propoxy]carbonylamino}-3-(4-chlorophenyl)butanoic acid, a pharmaceutically acceptable salt thereof, or a pharmaceutically acceptable solvate of any of the foregoing.

4. The oral dosage form of claim 1, wherein the combination comprises a first particle population (a) having a first release profile that is independent of the solution pH, and a second particle population (a) having a second release profile that is independent of the solution pH, wherein the second release profile is different than the first release profile.

5. The oral dosage form of claim 1 wherein the combination comprises a first particle population (b) having a third release profile that is dependent on the solution pH, and a second particle population (b) having a fourth release profile that is dependent on the solution pH, wherein the fourth release profile is different than the third release profile.

6. The oral dosage form of claim 1, wherein the concentration of (R)-baclofen does not exceed a concentration that causes moderate sedation and impairment of motor activity in the patient at any time after the dosage form is orally administered to the patient.

7. The oral dosage form of claim 1, wherein the continuous time period is chosen from at least about 4 hours, at least about 8 hours at least about 12 hours, at least about 16 hours, at least about 20 hours, and at least about 24 hours.

8. The oral dosage form of claim 1, wherein the therapeutically effective concentration of (R)-baclofen ranges from about 50 ng/mL to about 1,000 ng/mL.

9. The oral dosage form of claim 1, wherein the dosage form comprises a mass equivalent of (R)-baclofen ranging from about 0.1 mg to about 100 mg.

10. The oral dosage form of claim 1, wherein the dosage form is chosen from a once-daily dosage form and a twice-daily dosage form.

11. The oral dosage form of claim 10, wherein the once-daily dosage form comprises a mass equivalent of (R)-baclofen ranging from about 0.5 mg to about 50 mg.

12. An oral pharmaceutical dosage form of (3R)-4-{[(1S)-2-methyl-1-(2-methylpropanoyloxy)propoxy]carbonylamino}-3-(4-chlorophenyl)butanoic acid, a pharmaceutically acceptable salt thereof, or a pharmaceutically acceptable solvate of any of the foregoing, which when administered orally to a patient provides a therapeutically effective concentration of (R)-baclofen in the plasma of the patient for a continuous period of time after the dosage form is orally administered to the patient, wherein the concentration of (R)-baclofen in the plasma of the patient does not exceed a minimum adverse concentration at any time after the dosage form is orally administered to the patient.

13. The dosage form of claim 12, wherein the continuous time period is chosen from at least about 4 hours, at least about 8 hours, at least about 12 hours, at least about 16 hours, at least about 20 hours, and at least about 24 hours.

14. An oral dosage form comprising a plurality of pH-independent release particles comprising a (R)-baclofen prodrug of Formula (I), which following oral administration to a human patient provides a blood (R)-3-amino-3-(4-chlorophenyl)butanoic acid concentration characterized by:
a Cₘₐₓ/C₁₂ ratio ranging from about 1 to about 6;
a Cₘₐₓ/dose ratio ranging from about 1.25 (10⁶·mL)⁻¹ to about 3.25 (10⁶·mL)⁻¹; and
an AUC_{inf}/dose ratio ranging from about 13 (hr/10⁶·mL) to about 33 (hr/10⁶·mL).

15. The oral dosage form of claim 14, wherein each of the particles comprises (3R)-4-{[(1S)-2-methyl-1-(2-methylpropanoyloxy)propoxy]carbonylamino}-3-(4-chlorophenyl)butanoic acid, pharmaceutically acceptable salt thereof, or a pharmaceutically acceptable solvate of any of the foregoing; and at least one pH independent release polymer.

16. The oral dosage form of claim 15, wherein the at least one pH independent release polymer comprises an ammonioalkyl methacrylate copolymer.

17. The oral dosage form of claim 15, wherein the (3R)-4-{[(1S)-2-methyl-1-(2-methylpropanoyloxy)propoxy]carbonylamino}-3-(4-chlorophenyl)butanoic acid is present in a therapeutically effective amount.

18. The oral dosage form of claim 15, wherein the (3R)-4-{[(1S)-2-methyl-1-(2-methylpropanoyloxy)propoxy]carbonylamino}-3-(4-chlorophenyl)butanoic acid is present in an amount ranging from about 1 mg-equivalent to about 100 mg-equivalent of (R)-3-amino-3-(4-chlorophenyl)butanoic acid.

19. The oral dosage form of claim 15, wherein release of the (3R)-4-{[(1S)-2-methyl-1-(2-methylpropanoyloxy)propoxy]carbonylamino}-3-(4-chlorophenyl)butanoic acid from the oral dosage form exhibits the following in vitro dissolution profile in 10 mM monobasic potassium phosphate buffer at pH 7.4 at 37° C. agitated at 75 rpm (USP, Type II):
from about 35% to about 45% of the (3R)-4-{[(1S)-2-methyl-1-(2-methylpropanoyloxy)propoxy]carbonylamino}-3-(4-chlorophenyl)butanoic acid is released within about 4 hours;
from about 60% to about 80% of the (3R)-4-{[(1S)-2-methyl-1-(2-methylpropanoyloxy)propoxy]carbonylamino}-3-(4-chlorophenyl)butanoic acid is released within about 7.6 hours; and
from about 85% to about 95% of the (3R)-4-{[(1S)-2-methyl-1-(2-methylpropanoyloxy)propoxy]carbonylamino}-3-(4-chlorophenyl)butanoic acid is released within about 13 hours.

20. The oral dosage form of claim 14, which following oral administration to the human patient provides an oral bioavailability of (R)-3-amino-3-(4-chlorophenyl)butanoic acid ranging from about 20% to about 72%.

21. The oral dosage form of claim 14, which when administered orally to a patient provides a (R)-baclofen plasma concentration profile substantially as shown in FIG. 6.

22. The oral dosage form of claim 14, which when administered orally to a patient provides a (R)-baclofen plasma concentration profile that is bioequivalent to the profile shown in FIG. 6.

23. A method of treating a disease in a patient, comprising orally administering to a patient in need of such treatment at least one dosage form of any one of claims 1, 12, and 14, wherein the disease is chosen from spasticity, gastro-esophageal reflux disease, emesis, cough, narcotic addiction or abuse, alcohol addiction or abuse, nicotine addiction or abuse, neuropathic pain, and musculoskeletal pain.
